(19) United States
(12) Patent Application Publication
(10) Pub. No.: US 2025/0303148 A1
(43) Pub. Date: Oct. 2, 2025

(54) DELIVERY AND RETRIEVAL SYSTEM FOR A MEDICAL DEVICE

(71) Applicant: Medtronic, Inc., Minneapolis, MN (US)

(72) Inventors: Ronald A. Drake, St. Louis Park, MN (US); Lester O. Stener, Hudson, WI (US)

(21) Appl. No.: 18/866,770

(22) PCT Filed: May 24, 2023
(86) PCT No.: PCT/US2023/067400

§ 371 Date: Nov. 18, 2024

Related U.S. Application Data
(60) Provisional application No. 63/365,669, filed on Jun. 1, 2022.

(51) Int. Cl. A61N 1/05

(52) U.S. Cl. CPC A61N 1/057; A61N 2001/0578

(57) ABSTRACT

A medical system configured to impart a torque to a medical device within a patient. The medical system includes a driver including a driver body supporting a head section. The medical system includes a snare configured to engage the medical device. In examples, the head section defines a protrusion configured to insert into a device recess or a device slot of the implantable medical device to transfer the torque to the medical device. In examples, the a snare surface of the snare is configured to engage the medical device to transfer the torque to the medical device. In examples, the medical device includes an attachment member configured to engage or disengage tissue when the medical device rotates. The medical system may include a delivery catheter configured to deliver and/or retrieve the head section, intermediate member, and medical device.

### TECHNICAL FIELD

This application claims the benefit of U.S. Provisional Patent Application Ser. No. 63/365,669, filed Jun. 1, 2022, the entire content of which is incorporated herein by reference.

### TECHNICAL FIELD

This disclosure is related to system for delivery and/or retrieval of implantable medical devices.

### BACKGROUND

Various types of implantable medical devices have been implanted for treating or monitoring one or more conditions of a patient. Such implantable medical devices may be adapted to allow medical devices to monitor and/or treat conditions or functions relating to heart, muscle, nerve, brain, stomach, endocrine organs or other organs and their related functions. The implantable medical devices may be implanted at target locations selected to detect a physiological condition of the patient and/or deliver one or more therapies. For example, implantable medical devices may be delivered to locations within an atrium or ventricle of a heart to sense intrinsic cardiac signals and deliver pacing or antitachyarrhythmia shock therapy.

Some implantable medical devices are sized to be completely implanted within one of the chambers of the heart and/or another anatomical volume of the patient to detect a physiological condition and/or deliver one or more therapies. Such implantable medical devices may utilize delivery and/or retrieval systems to allow a clinician to navigate the implantable medical device (e.g., through vasculature of the patient) to the target location, and/or to retrieve the implantable medical device from the patient. In some examples, the implantable medical device may include one or more anchoring components intended to engage tissues at the target location (e.g., for implantation) and/or disengage from tissue at the target location (e.g., for retrieval).

### SUMMARY

The disclosure describes a medical system configured to deliver, position, retrieve, and/or otherwise re-orient an implantable medical device (“IMD”) within an anatomical volume (e.g., a chamber of a heart) within a patient. The medical system includes a driver including a driver body and a head section supported substantially at a distal end of the driver body. The driver is configured to engage (e.g., contact) the head section and the IMD (e.g., a retrieval structure of the IMD). The medical system further includes a snare configured to translate through a lumen defined by the driver to engage and exert a force on the IMD. In examples, the snare is configured to exert the force on the IMD when a snare loop of the snare substantially constricts around the IMD and a proximal force is exerted on a distal portion of the snare (e.g., when a tension is applied to the snare). The snare is configured to cause the head section to exert a contact force on the IMD when head section engages the IMD and the snare exerts the force.

The head section defines a protrusion configured to insert into a device recess or a device slot defined by the IMD. The protrusion is configured to transfer a torque from the driver body to the ID when the snare causes the head section to exert the contact force and the protrusion inserts within the device recess or device slot. The head section is configured to substantially roll and/or slide over the ID under the influence of the torque from the driver body until the protrusion inserts within the device recess or device slot. In examples, the medical system includes a delivery catheter configured to deliver and/or retrieve the head section, the intermediate member, and/or the implantable medical device through vasculature of the patient.

In an example, a medical system comprises: a driver configured to impart a torque on an implantable medical device within an anatomical volume defined by a body of a patient, the driver including a driver body and a head section supported by the driver body, wherein the driver defines a lumen extending through at least a portion of the driver body and at least a portion of the head section, the lumen opening to a lumen opening defined by the head section, and wherein the head section defines a protrusion configured to insert within a device recess or a device slot of the implantable medical device; and a snare configured to slidably translate within the lumen and through the lumen opening to engage the implantable medical device, wherein the snare is configured to cause the head section to exert a contact force on the implantable medical device when the snare engages the implantable medical device and imparts a force on the implantable medical device, and wherein the head section is configured to transfer the torque from the driver body to the implantable medical device when the snare causes the contact force and the protrusion inserts within the device recess or the device slot.

In an example, a medical system comprises: a driver configured to impart a torque on an implantable medical device within an anatomical volume defined by a body of a patient, the driver including a driver body and a head section supported by the driver body, wherein the driver defines a lumen extending through at least a portion of the driver body and at least a portion of the head section, the lumen opening to a lumen opening defined by the head section; and a snare configured to slidably translate within the lumen and through the lumen opening to engage the implantable medical device, wherein the snare is configured to cause the head section to exert a contact force on the implantable medical device when the snare engages the implantable medical device and imparts a force on the implantable medical device, wherein the head section is configured to deform and generate a frictional force with the implantable medical device when the snare causes the contact force and the driver body imparts a torque on the head section, and wherein the head section is configured to transfer the torque to the implantable medical device when the head section generates the frictional force.

In an example, a medical system configured to impart a torque on an implantable medical device within an anatomical volume defined by a body of a patient, comprising: a snare comprising a snare loop a snare comprising a snare loop configured to at least partially surround an implantable medical device within an anatomical volume defined by a body of a patient, wherein the snare loop includes a snare surface defining a textured surface; a driver including a driver body defining a distal portion and a proximal portion, the driver body defining a lumen extending through the distal portion and the proximal portion, and the distal portion defining a lumen opening that opens to the lumen, wherein the snare extends through the driver lumen and the lumen opening, wherein the driver body is configured to position between the snare and the implantable medical device when the snare at least partially surrounds the implantable medical device and the driver body translates in a distal direction relative to the snare, wherein the snare is configured to constrict around the implantable medical device to exert a snare-imparted force on the implantable medical device when the snare loop at least partially surrounds the implantable medical device and a proximal force is exerted on the snare (e.g., when a tension is applied to the snare). The snare-imparted force may being a portion of the proximal force, wherein the snare surface is configured to frictionally engage the implantable device using the textured surface when the snare loop exerts the snare-imparted force on the implantable medical device, wherein the driver body is configured to exert a force on the snare when the snare surface frictionally engages the implantable medical device, and wherein the snare is configured to impart a torque to the implantable medical device using the frictional engagement when the driver exerts the force on the snare.

In an example, a technique comprises: engaging an implantable medical device, using a snare extending through a lumen and a lumen opening defined by a driver body of a driver, to cause a head section of the driver to exert a contact force on the implantable medical device; imparting a torque, using the driver body, on the head section, wherein the head section defines a protrusion configured to insert within a device recess or a device slot of the implantable medical device; and transferring, using the head section, the torque from the head section to the implantable medical device when the snare causes the contact force and the protrusion inserts within the device recess or the device slot.

In an example, a technique comprises: exerting, using a driver including a driver body defining a lumen and a lumen opening, a force on a snare when a snare surface of the snare frictionally engages an implantable medical device, wherein the snare extends through the lumen and the lumen opening, and wherein the driver is positioned between the snare and the implantable medical device, and wherein the snare is configured to constrict around the implantable medical device to exert a snare-imparted force on the implantable medical device when a snare loop of the snare at least partially surrounds the implantable medical device and a proximal force is exerted on the snare, the snare-imparted force being a portion of the proximal force; and imparting, using the snare, a torque on the implantable medical device when the driver exerts the force on the snare.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system and delivery catheter within a heart.

FIG. 2 is a perspective illustration of an example medical system including a driver and snare.

FIG. 3 is a perspective illustration of an example medical system including a driver and a snare positioned relative to an implantable medical device.

FIG. 4 is a perspective illustration of a head section engaging an implantable medical device.

FIG. 5 is a schematic diagram of an example medical system including a driver, a snare, and a delivery catheter shown in conjunction with an example implantable medical device.

FIG. 6 is a longitudinal cross-sectional diagram of the example medical system of FIG. 5.

FIG. 7 is a schematic diagram of the example medical system of FIG. 5 engaging the example implantable medical device.

FIG. 8 is a transverse cross-sectional diagram of the example medical system of FIG. 5 with a head section in a first position relative to the implantable medical device.

FIG. 9 is a transverse cross-sectional diagram of the example medical system with a head section in a second position relative to the implantable medical device.

FIG. 10 is a transverse cross-sectional diagram of the example medical system with a head section in a third position relative to the implantable medical device.

FIG. 11 is a transverse cross-sectional diagram of the example medical system with a head section in a fourth position relative to the implantable medical device.

FIG. 12 is a schematic illustration of driver and a snare of the example medical system.

FIG. 13 is a cross-sectional diagram of the example medical system of FIG. 12.

FIG. 14 is a schematic illustration of a snare of the medical system engaging a medical device.

FIG. 15 is a cross-sectional diagram of the example medical system of FIG. 14.

FIG. 16 is a perspective illustration of an example medical device.

FIG. 17 is a perspective illustration of an example medical device including a pin member.

FIG. 18 illustrates an example technique for transferring a torque to an implantable medical device.

FIG. 19 illustrates an example technique for transferring a torque to an implantable medical device using a snare surface.

### DETAILED DESCRIPTION

This disclosure describes a medical system configured to deliver, position, and/or retrieve an implantable medical device (“IMID”) within an anatomical volume (e.g., a chamber of a heart) within a patient. The medical system is configured to receive a torque (e.g., from a clinician) and impart the torque to IMD to implant, retrieve, re-position, and/or re-orient the IMD in the anatomical volume. In examples, the medical system is configured to enable a rotation of the IMD around a device axis when the medical system imparts the torque. The rotation of the implantable medical device may cause an attachment member of the implantable medical device to engage tissues at a target site, disengage tissues at the target site, and/or otherwise cause a re-orientation of the medical device within the patient.

The medical system includes a driver configured to receive the torque (e.g., from the clinician) and impart the torque to the IMD, The driver includes a driver body (e.g., an elongate body) and a head section. In examples, the head section is substantially at a distal end of the driver (“driver distal end”). The head section is configured to engage (e.g., contact) the IMD (e.g., a retrieval structure of the IMD) when the medical system imparts the torque to the IMD. The medical system further includes a snare configured to extend through a lumen of the driver (“driver lumen”) to engage and exert a force on the IMD. The medical system is configured such that exerting a force on the IMD using the snare may cause the head section to exert a contact force against the IMD. For example, the head section may substantially translate over the snare to position between a first portion of the snare (“first snare portion”) and the IMD. When the snare engages and exerts the force on the IMD using a second portion of the snare (e.g., a second snare portion distal to the first snare portion), the first snare portion may substantially place the head section in compression between the first snare portion and the IMD, such that the head section exerts the contact force (e.g., a reaction force) against the IMD. The contact force may be, for example, a force imparted by the head section against the IMD, such that the contact force substantially causes the head section to maintain contact with the IMD. In examples, the head section is configured to exert the contact force against a retrieval structure of the IMD (“IMD retrieval structure”), such as against a perimeter defined by the IMD retrieval structure.

In some examples, the head section defines a protrusion (e.g., a corner or other protrusion) configured to insert into a device recess or device slot of the IMD (e.g., the IMD retrieval structure) when the snare causes the head section to exert the contact force. The protrusion may be configured to transfer a torque from the driver body (e.g., a torque imparted by a clinician) to the IMD when the protrusion inserts into the device slot or device recess. In some examples, instead of or in addition to the protrusion, the snare (e.g., a snare loop) includes a snare surface configured to frictionally engage the IMD to transfer the torque from the driver body. The snare surface may define a textured surface (e.g., a surface roughness and/or surface undulation) configured to substantially grip the IMD when the snare loop tightens (e.g., constricts) around the IMD. The frictional engagement may generate the torque on the IMD snare surface when the driver (e.g., the driver body and/or head section) exerts a force on the snare (e.g., a force substantially perpendicular to the contact force exerted by the head section).

When the head section defines a protrusion configured to insert into a device recess or device slot, the medical system may primarily use the protrusion to transfer a torque from the driver body to the IMD. When the snare includes a snare surface defining a textured surface configured to frictionally engage the IMD, the medical system may primarily use the snare surface to transfer the torque from the driver body to the IMD. Hence, the medical system may be configured to primarily use the protrusion to transfer a torque, be configured to primarily use the snare surface to transfer the torque, or be configured to use both the protrusion and the snare surface to transfer the torque.

The snare is configured to translate through the driver lumen and exert a force on the IMD when a snare loop of the snare substantially tightens (e.g., constricts) around the IMD and a proximal force is exerted on a distal portion of the snare (e.g., when a tension is applied to the snare). The snare is configured such that, when the snare extends through the driver lumen and exerts the force on the IMD, the snare causes the head section to exert the contact force on the IMD when head section engages the IMD. In examples, the head section is configured to position substantially between the snare (e.g., the first snare portion) and the IMD when the snare exerts the force on the IMD and causes the head section to exert the contact force on the IMD. The driver may be configured such that, when the snare extends through the driver lumen and exerts the force on the IMD, the snare substantially presses the head section against the IMD, causing the head section to exert the contact force on the IMD.

For example, the driver lumen may extend through at least a portion of the driver body and through the head section to a lumen opening defined by the head section. The snare may extend through the lumen opening when the snare engages the IMD and exerts the force on the IMD. Thus, when the snare extends through the lumen opening and the head section positions between the snare and the IMD, the force exerted by the snare on the IMD substantially presses the head section against the IMD, causing the head section to exert the contact force on the IMD. In examples, the driver lumen defines a longitudinal axis extending through the lumen opening, and the lumen opening is configured to substantially redirect a force exerted on the snare (e.g., by a clinician) from a first direction substantially parallel to the longitudinal axis to a second direction defining an oblique angle to the longitudinal axis. The redirected force may act on the IMD to substantially pull the IMD into the head section, causing the head section to exert the contact force on the IMD.

In examples wherein the head section defines a protrusion, the head section is configured to transfer torque from the driver body to the IMD when the protrusion inserts into a device recess or device slot defined by the IMD and the head section exerts the contact force on the IMD. In examples, the head section defines a head perimeter substantially surrounding the longitudinal axis extending through the lumen opening, and the protrusion (e.g., a corner defined by the head section) defines some portion of the head perimeter. The head section may be configured to substantially roll or slip around a structure perimeter defined by the IMD to cause the protrusion to insert within the device recess or device slot of the IMD. In examples, the structure perimeter substantially surrounds a device axis defined by the IMD.

For example, when a torque is imparted on the driver body (e.g., by a clinician), the torque may cause the driver body and the head section to rotate about the longitudinal axis defined by the driver lumen. When the snare causes the head section to exert the contact force against the IMD, the rotation may cause the driver body and the head section to rotate relative to the IMD, such that the head section substantially rolls or slips around the structure perimeter. The driver body and the head section may experience an angular displacement around the device axis relative to the IMD when the head section substantially rolls or slips around the structure perimeter. The driver may be configured such that, when the protrusion of the head section inserts into the device recess or device slot of the IMD, the driver body, the head section, and the IMD lock into substantially stationary relative positions, such that the continued torque exerted on the driver body causes the protrusion to transfer at least some portion of the torque to the IMD, causing rotation of the IMD about the device axis.

Stated similarly, when the driver body, the head section, and the IMD lock into substantially the stationary relative positions and continued torque on the driver body continues to cause the rotation of the driver body and the head section about the longitudinal axis defined by the driver lumen, the longitudinal axis may begin to substantially orbit around the device axis of the IMD to accommodate the continued rotation of the driver body and the head section. With the driver body, the head section, and the IMD locked into substantially stationary relative positions, the orbiting of the longitudinal axis around the device axis causes the protrusion to impart at least some portion of the torque to the IMD, causing rotation of the IMD about the device axis.

Hence, when the snare causes the head section to exert the contact force against the ID, the torque imparted on the driver body (e.g., by a clinician) may cause the protrusion of the head section to insert into the device recess or device slot of the IMD, and may cause the head section to transfer torque to the IMD, causing rotation of the ID about the device axis. In examples, the ID includes an attachment member (e.g., a helix) configured to engage tissue or disengage from tissue based on the rotation of the ID. The rotation of the ID about the device axis may cause the attachment member of the implantable medical device to engage tissues at a target site, disengage tissues at the target site, and/or otherwise cause a re-orientation of the medical device within the patient.

The snare is configured to slidably translate within the driver lumen, such that a clinician may cause the snare to translate proximally and/or distally within the driver lumen, and/or cause the driver body and head section to translate relative to the snare. For example, the snare may be configured to extend through the driver lumen and distal to the head section such that the snare loop may engage the IMD (e.g., the ID retrieval structure) when the IMD is distal to the head section. A clinician may cause the driver body and the head section to translate distally over the snare when the snare loop is engaged with the IMD, such that the snare substantially guides the head section toward the IMD retrieval structure.

As used herein, a contact force exerted from the head section to the IMD retrieval structure may include one or more action forces and/or one or more reaction forces. For example, the contact force may include an action force imparted by head section to the IMD retrieval structure when the head section exerts the distally-directed force. The contact force may include a reaction force generated by the head section in response to the snare exerting the proximally-directed force on the IMD and the IMD transmitting the proximally directed force to the head section. Hence, the medical system is configured such that a clinician may control the contact force exerted from the head section to the ID retrieval structure using the driver and the snare in combination or individually.

In some examples, in addition to or instead of transferring torque to the IMD using a head section defining a head section protrusion, the medical system is configured to transfer torque to the IMD using the snare surface defined by the snare. In examples, the snare loop of the snare defines the snare surface. The snare surface may define a textured surface having a texture defined by, for example, a surface roughness or an undulating surface, configured to frictionally engage the IMD when the snare loop engages (e.g., surrounds a portion of) the ID. The driver body and head section may translate distally toward the IMD relative to the snare to position between the snare (e.g., the first snare portion) and the IMD when the snare loop engages the ID. For example, the driver body and head section may translate substantially over the snare as the snare extends through the driver lumen, such that the snare substantially guides the head section to a position between the first snare portion and the IMD (e.g., the IMD retrieval structure).

As before, the snare may extend through the lumen opening when the snare engages the IMD and exerts the force on the IMD. The force exerted by the snare on the ID may substantially press the head section against the IMD, causing the head section to exert the contact force on the IMD. The lumen opening may substantially redirect a force exerted on the snare (e.g., by a clinician) from a first direction substantially parallel to the longitudinal axis to a second direction defining an oblique angle to the longitudinal axis, such that the redirected force substantially pulls the IMD into the head section.

The snare (e.g., the first snare portion) may substantially place the head section in compression between the first snare portion and the IMD when the snare substantially pulls the ID into the head section. The head section may be configured to exert a first reaction force on the first snare portion in response to the compression. Likewise, when the second snare portion exerts the force on the IMD, the snare may be configured such that the IMD exerts a second reaction force on the second snare portion. For example, the ID may exert the second reaction force in response to a snare-imparted force exerted on the ID by the snare. The first reaction force and the second reaction force may act in substantially opposite directions, such that the first reaction force and the second reaction force place the snare in tension. The snare may be configured such that increasing a proximally-directed force on a proximal portion of the snare causes an increase in the snare-imparted force and the corresponding second reaction force, increasing a frictional force between the snare surface and the ID. The medical system may be configured such that a tension applied to the snare (e.g., a proximal force on the snare imparted by a clinician) generates a sufficient frictional force such that the snare acts to impart a torque to the IMD when the driver body exerts a force substantially perpendicular to the frictional force on the snare (e.g., the second snare portion).

For example, when the snare surface generates a sufficient snare-imparted force on the IMD, a clinician may impart a force substantially perpendicular to the snare imparted contact force on a proximal portion of the driver body. The driver body may transmit the imparted force to the head section, causing the head section to exert the imparted force to the second snare portion compressing the head section between the second snare portion and the IMD. The imparted force may cause the second snare portion to revolve around an axis defined by the IMD (“device axis.”), such that the textured surface of the snare surface and the snare-imparted force generate a frictional force between the snare surface and the IMD. The frictional force and the rotation of the second snare portion around the device axis may impart a torque to the IMD, causing the IMD to substantially revolve about the device axis. Hence, a clinician may impart a torque to the IMD by sufficiently increasing the snare-imparted force using the snare while imparting a force substantially perpendicular to the snare-imparted normal force using the driver.

The IMD may include an IMD retrieval structure defining a retrieval structure perimeter substantially surrounding the device axis of the IMD. The snare (e.g., the second snare portion) may be configured to substantially surround the retrieval structure perimeter when the snare frictionally engages the IMD. In examples, an IMD retrieval structure of the IMD defines a retrieval structure protrusion (e.g., a corner) configured to increase the frictional engagement between the IMD and the snare surface. The retrieval structure protrusion may define some portion of the retrieval structure perimeter. For example, the retrieval structure may be configured such that the retrieval structure perimeter defines a polygon (e.g., a hexagon) defining a plurality of protrusions. The protrusions may be configured to increase the frictional engagement between the IMD and the snare surface, and/or substantially bear against the snare surface, such that the second snare portion transfers a torque to the IMD retrieval structure when the first snare portion rotates around the device axis.

In examples, the medical system may include a delivery catheter including a delivery receptacle. The delivery receptacle may define a delivery receptacle volume configured to receive the driver, the snare, and at least a portion of the IMD. The delivery catheter may define a delivery catheter lumen and a delivery lumen opening which opens to the delivery receptacle volume. The driver may be configured to slidably translate within the delivery catheter lumen and through the delivery lumen opening, such that relative movement between the driver and the delivery catheter may cause the driver, the snare, and/or at least the portion of the ID to position within the delivery receptacle volume and/or exit the delivery receptacle volume (via the delivery lumen opening). The delivery catheter may be configured to transition through the vasculature of a patient, such that the driver, the snare, and/or the IMD may be retrieved from and/or delivered to an anatomical volume of the patient (e.g., a heart chamber).

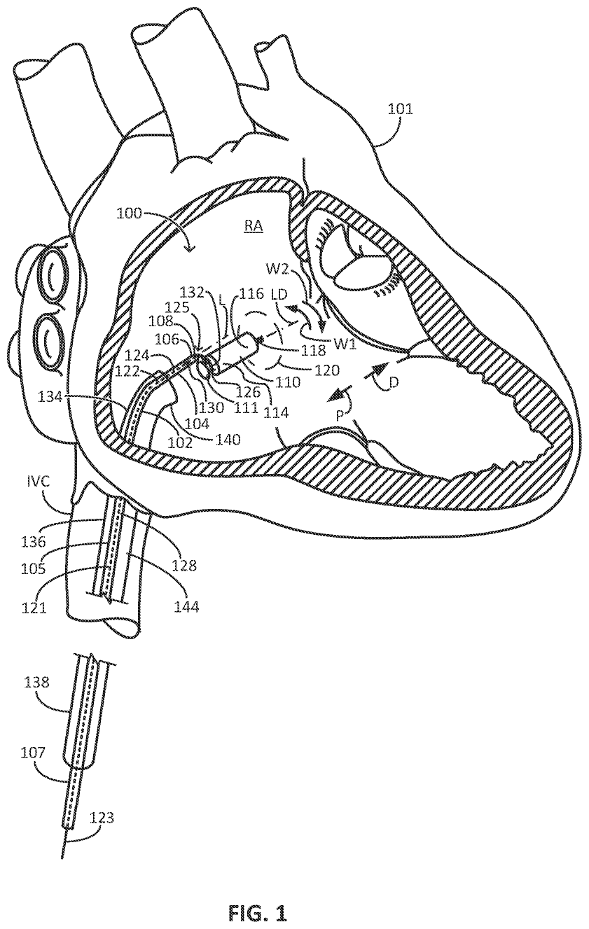

FIG. 1 is a conceptual diagram illustrating an example medical system 100 within a right atrium (“RA”) of a heart 101. Medical system 100 includes driver 102 including driver body 104. Driver body 104 may include a distal portion 105 (“driver body distal portion 105”) configured to be intracorporeal to the patient and a proximal portion 107 (“driver body proximal portion 107”) which may be extracorporeal to the patient when driver body distal portion 105 is intracorporeal. A head section 106 is supported at a distal end 108 of driver body 104 (“driver body distal end 108”). In some examples, head section 106 and driver body 104 may be substantially separate components. In some examples, head section 106 may be substantially contiguous with driver body 104, such that head section 106 and driver body 104 define a unified component.

Medical system 100 is configured to transfer a torque to an IMD 110 within an anatomical volume of a patient, such as the RA of heart 101. In examples, medical system 100 is configured to engage a proximal portion 114 of IMD 110 (“IMD proximal portion 114”) to transfer the torque. In examples, IMD proximal portion 114 includes a retrieval structure 111 (“IMD retrieval structure 111”). IMD retrieval structure 111 may be configured to engage with medical system 100 and/or another medical device to, for example, implant IMD 110 within an anatomical volume, retrieve IMD 110 from an anatomical volume, re-position IMD 110 within an anatomical volume, and/or re-orient IMD 110 within an anatomical volume. IMD 110 may include a distal portion 116 (“IMD distal portion 116”) opposite IMD proximal portion 114.

In examples, IMD 110 (e.g., IMD distal portion 116) supports an attachment member 118 configured to engage tissue within a target site 120 of an anatomical volume. Attachment member 118 may be supported in IMD distal portion 116. In some examples, attachment member 118 is configured (e.g., as a helix) such that rotation of IMD 110 about a device axis LD defined by IMD 110 causes attachment member to engage and/or disengage tissues with target site 120. For example, attachment member 118 may be configured such that rotation of IMD 110 in a first rotational direction W1 about device axis LD causes attachment member 118 to engage (or alternately, disengage from) tissues within target site 120. Attachment member 118 may be configured such that rotation of IMD 110 about device axis LD in a second rotational direction W2 substantially opposite first rotational direction W1 causes attachment member 118 to disengage from (or alternately, engage) tissues within target site 120. In examples, IMD 110 includes one or more components (e.g., a communication antenna, a sensor, or another component) configured to rotate around and or revolve about device axis LD when IMD 110 rotates about device axis LD. Medical system 100 may cause IMD 110 to rotate about device axis LD to cause one or more of the components to substantially establish a specific orientation with respect to the anatomy of the patient, another device implanted within or worn by the patient, another device external to the patient, and/or other devices.

Driver body 104 is configured to receive a torque (e.g., from a clinician) and transfer the torque to head section 106. Head section 106 is configured to engage IMD 110 (e.g., IMD retrieval structure 111) and transfer the torque to IMD 110. Driver body 104 may be a substantially elongate body. In examples, head section 106 is configured to position against (e.g., contact) IMD 110 to transfer the torque. Medical system 100 may be configured to cause head section 106 to exert a contact force against IMD 110 (e.g., IMD retrieval structure 111) sufficient to minimize and/or substantially prevent slippage of head section 106 when driver body 104 causes head section 106 to rotate around device axis LD. When the contact force is sufficient to minimize and/or substantially prevent the slippage, the rotation of head section 106 around device axis LD may cause IMD 110 to revolve about device axis LD. Hence, medical system 100 is configured to mechanically couple driver 102 and IMD 110 using at least head section 106, such that a torque imparted to driver body 104 may cause rotation of IMD 110 in first rotational direction W1 and/or second rotation direction W2 to, for example, implant IMD 110, retrieve IMD 110, re-position IMD 110, and/or re-orient IMD 110 within an anatomical volume such as the RA.

Medical system includes a snare 122 configured to cause head section 106 to exert the contact force on IMD 110. Snare 122 may include a body 124 (“snare body 124”) and a loop 126 (“snare loop 126”) at a distal end of snare body 124. Snare loop 126 is configured to engage IMD 110 (e.g., IMD retrieval structure 111) to, for example, guide head section 106 toward IMD retrieval structure 111 and/or control a contact force between head section 106 and IMD 110 (e.g., IMD retrieval structure 111). In examples, driver body 104 defines a lumen 128 (“driver lumen 128”) and a lumen opening (driver lumen opening 125 substantially at driver distal end 108. Snare 122 may be configured to slidably translate within driver lumen 128, such that a clinician may cause snare 122 to translate proximally (e.g., in the direction P) and/or distally (e.g., in the direction D) within driver lumen 128, and/or cause driver 102 and head section 106 to translate proximally and/or distally relative to snare 122. For example, snare 122 may be configured to extend through driver lumen 128 and distal to head section 106 to engage IMD 110. A clinician may cause driver 102 and head section 106 to translate distally over snare 122 (e.g., over snare body 124 and/or snare loop 126) when snare loop 126 is engaged with IMD 110, such that snare 122 substantially guides head section 106 toward IMD retrieval structure 111.

For example, head section 106 may substantially translate over snare 122 to position between a first portion 130 of snare 122 (“first snare portion 130”) and IMD 110. When the snare engages and exerts a force (e.g., a force in the proximal direction P) on IMD 110 using a second portion 132 of snare 122 (“second snare portion 132”), first snare portion 130 may substantially place head section 106 in compression between first snare portion 130 and IMD 110 (e.g., IMD retrieval structure 111), such that head section 106 exerts the contact force (e.g., a reaction force) against IMD 110. Snare 122 is configured to translate through driver lumen 128 to exert the force on the IMD when snare loop 126 substantially tightens (e.g., constricts) around IMD 110 and a proximal force is exerted (e.g., by a clinician) on a distal portion 121 of snare 122 (“snare distal portion 121”). In examples, snare distal portion 121 is configured to be intracorporeal to the patient (e.g., via driver lumen 128). Snare 122 may define a proximal portion 123 (“snare proximal portion 123”), which may be extracorporeal to the patient when snare distal portion 121 is intracorporeal. In examples, driver lumen 128 defines a longitudinal axis L extending through driver lumen opening 125, and driver lumen opening 125 is configured to substantially redirect a force exerted on snare 122 (e.g., by a clinician) from a first direction substantially parallel to longitudinal axis L to a second direction defining an oblique angle to longitudinal axis L, such that the redirected force acts on the IMD 110 (e.g., IMD retrieval structure 111) to substantially pull IMD 110 into head section 106, causing head section 106 to exert the contact force on IMD 110. In examples, the contact force exerted by head section 106 includes a force component acting on IMD 110 in a direction substantially perpendicular to longitudinal axis L.

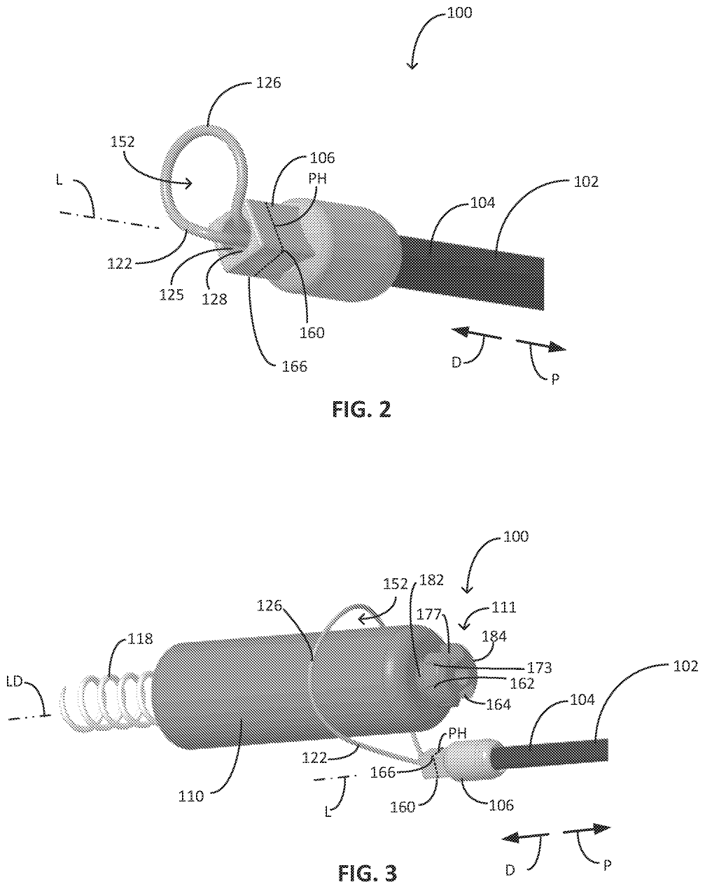

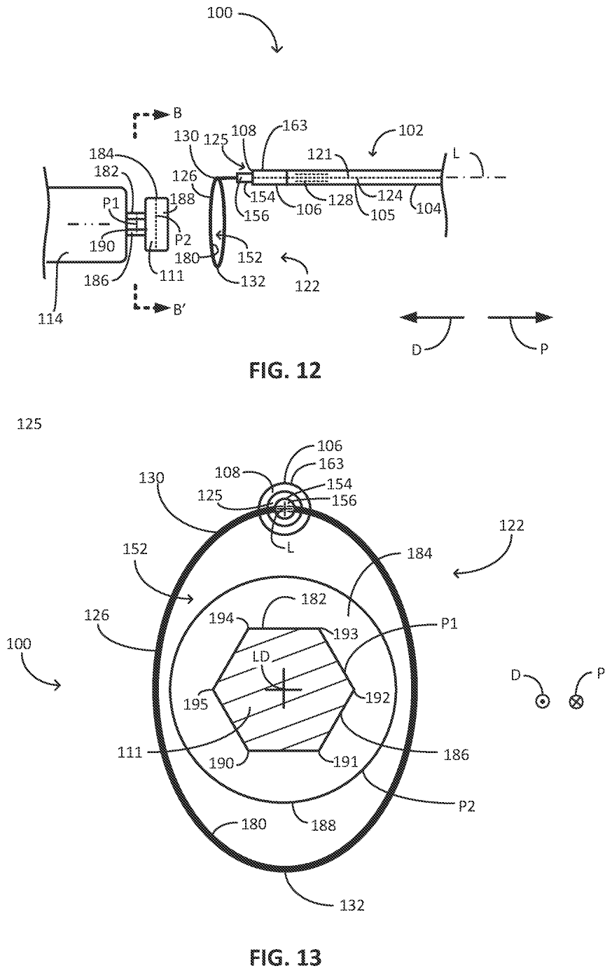

In examples, head section 106 defines a protrusion (e.g., protrusion 160 (FIGS. 2-4, 8-11)) configured to insert into a device recess (e.g., device recess 162 (FIGS. 2-11) or a device slot (e.g., device slot 164 (FIGS. 2-11)) of IMD 110 (e.g., IMD retrieval structure 111) when snare 122 causes head section 106 to exert the contact force on IMD 110. The protrusion may be configured to transfer a torque from driver body 104 (e.g., a torque imparted by a clinician) to IMD 110 when the protrusion inserts into the device slot or device recess. In examples, the torque is applied to both driver body 104 and snare body 124 such that, for example, driver body 104 and snare body 124 rotate substantially around device axis LD. In some examples, instead of or in addition to the protrusion, snare 122 (e.g., snare loop 126) includes a snare surface (e.g., snare surface 180 (FIGS. 12-15)) configured to frictionally engage IMP 110 to transfer the torque from driver body 104. The snare surface may define a textured surface defining a texture (e.g., a surface roughness and/or surface undulation) configured to substantially grip IMD 110 when snare loop 126 tightens (e.g., constricts) around IMD 110. The frictional engagement may generate the torque on the snare surface when driver 102 (e.g., driver body 104 and/or head section 106) exerts a force on snare 122 (e.g., a force substantially perpendicular to longitudinal axis L). Medical system 100 may be configured to primarily use the protrusion to transfer a torque, be configured to primarily use the snare surface to transfer the torque, or be configured to use both the protrusion and the snare surface to transfer the torque. In examples, IMD retrieval structure 111 defines a retrieval structure protrusion (e.g., retrieval structure protrusion 190 (FIGS. 12-15)) configured to increase the frictional engagement between IMD 110 and the snare surface.

Medical system 100 may include a delivery catheter 134 configured to retrieve head section 106, snare 122, and/or IMD 110 from an anatomical volume of the patient (e.g., the RA). In examples, delivery catheter 134 is configured to deliver head section 106, snare 122, and/or IMD 110 to an anatomical volume of the patient. Delivery catheter 134 is illustrated as transparent in FIG. 1 for clarity. Delivery catheter 134 may include a distal portion 136 (“delivery catheter distal portion 136”) configured to be intracorporeal to the patient and a proximal portion 138 (“delivery catheter proximal portion 138”) which may be extracorporeal to the patient when delivery catheter distal portion 136 is intracorporeal. In examples, delivery catheter 134 is configured to deliver and/or retrieve head section 106, driver body 104, and/or IMD 110 using vasculature of a patient, such as an IVC or other vasculature leading to the anatomical volume.

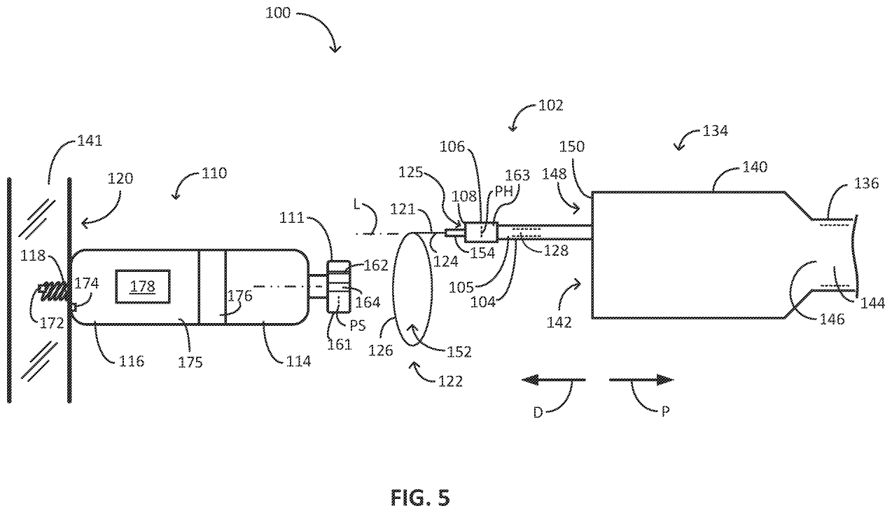

In examples, delivery catheter 134 includes a delivery receptacle 140 or receptacle defining a delivery receptacle volume (e.g., delivery receptacle volume 142 (FIGS. 5-7)) configured to receive head section 106, snare 122, and at least a portion of IMD 110. Delivery catheter 134 may define a lumen 144 (“delivery catheter lumen 144”) and a delivery lumen opening (e.g., delivery lumen opening 146 (FIGS. 5-7)) which opens to delivery receptacle volume 142. At least driver body 104 may be configured to slidably translate within delivery catheter lumen 144 and through the delivery lumen opening such that relative movement between driver body 104 and delivery catheter 134 may cause relative movement between head section 106 and/or IMD 110 and delivery catheter 134. Delivery receptacle 140 may define an opening (e.g., delivery receptacle opening 148 (FIGS. 5-7)) at a distal end of delivery receptacle 140 (e.g., delivery receptacle distal end 150 (FIGS. 5-7)). The delivery receptacle opening may be configured such that head section 106, snare 122, and at least a portion of IMD 110 may pass therethrough.

Delivery catheter 134 may be configured to retrieve IMD 110 when IMD 110 is anchored to tissues within target site 120 (e.g., anchored by attachment member 118). For example, delivery catheter 134 may be configured to transition through vasculature of a patient to position head section 106 and snare 122 in the proximity of IMD 110 when IMD 110 is anchored to tissues within target site 120. Delivery catheter 134 may be configured such that a force in the distal direction D on snare 122 (e.g., by a clinician) causes snare 122 to extend distal to the delivery receptacle opening of delivery receptacle 140, such that snare 122 (e.g., snare loop 126) may engage IMD 110. Delivery catheter 134 may be configured such that a force in the distal direction D on driver body 104 (e.g., exerted by a clinician) causes head section 106 to extend distal to the delivery receptacle opening of delivery receptacle 140, such that driver body 104 may translate over snare 122 to position head section 106 substantially between snare 122 (e.g., first snare portion 130) and IMD 110. Delivery catheter 134 (e.g., delivery receptacle 140) may be configured to receive head section 106, snare 122, and a portion of IMD 110 when head section 106 is positioned between snare 122 and IMD 110. For example, delivery catheter 134 may move in the distal direction D relative to head section 106, snare 122, and IMD 110 to receive head section 106, snare 122, and at least the portion of IMD 110. Delivery catheter 134 may be configured to remove head section 106, snare 122, and IMD 110 from an anatomical volume of the patient (e.g., the RA) once, for example, head section 106 and/or snare 122 have imparted a torque to IMD 110 causing attachment member 118 to disengage from tissues within target site 120. Delivery catheter 134, head section 106, snare 122, and IMD 110 may subsequently be withdrawn from the patient (e.g., via vasculature of the patient).

In examples, delivery catheter 134 is configured to position IMD 110 in proximity to target site 120 such that IMD 110 may be anchored to tissues within target site 120 (e.g., anchored by attachment member 118). For example, delivery catheter 134 may be configured to position head section 106 and snare 122 within delivery receptacle volume 142 when head section 106 is positioned between snare 122 and IMD 110. Delivery catheter 134 may be configured to traverse vasculature of the patient to position IMD 110 (e.g., attachment member 118) within or in proximity to target site 120. Medical system 100 (e.g., driver body 104, head section 106, and/or snare 122) may impart a torque to IMD 110 to cause attachment member 118 to engage tissues (e.g., tissue within target site 120) when attachment member 118 is within or in proximity to target site 120. Medical system 100 may be configured such that head section 106 and/or snare 122 may be disengaged from IMD 110 as IMD 110 remains anchored to tissues within or in proximity to target site 120. Delivery catheter 134, head section 106, and/or snare 122 may subsequently be withdrawn from the patient (e.g., via vasculature of the patient).

Although the examples herein discuss delivery, retrieval, and/or positioning of IMD 110 within the RA of heart 101, medical system 100 may be configured to position IMD 110 in any of the other chambers of heart 101 and/or in other anatomical volumes of a patient in a like manner as that described for the RA of heart 101. Further, although the examples herein discuss attachment member 118 defining a helix, attachment member 118 may defines other structures, such as one or more elongated tines extending from, for example, IMD distal portion 116. Target site 120 may include an appendage of the RA, or the triangle of Koch region of the RA, or some other portion of heart 101, or some other location within a body of a patient.

FIG. 2 is a perspective view of a portion of medical system 100 with head section 106 defining a protrusion configured to insert into a device recess or device slot of IMD 110, including snare 122 and driver 102. Snare 122 extends through driver lumen 128 and driver lumen opening 125 defined by driver body 104. Head section 106 defines one or more protrusions (e.g., one or more corners) such as protrusion 160 and second protrusion 166 around a head perimeter PH defined by head section 106. Head perimeter PH may substantially surround longitudinal axis L extending through driver lumen 128 and driver lumen opening 125. In examples, head perimeter PH defines a polygon, such as a square or other polygon.

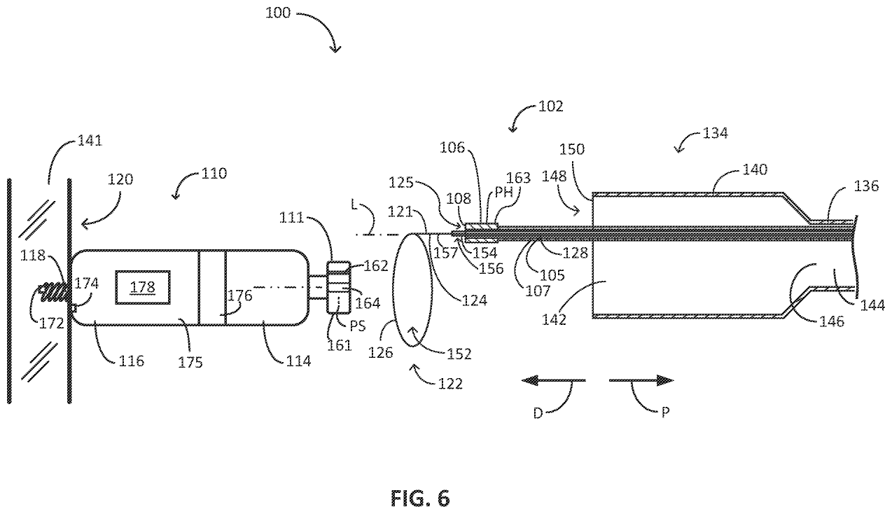

FIG. 3 is a perspective view of medical system 100 with snare 122 extended distal to driver 102 to engage IMD 110. Snare loop 126 and/or driver 102 may be translated relative to IMD 110 such that snare loop 126 substantially surrounds a portion of IMD 110 and device axis LD. Driver 102 and/or snare sheath 154 (FIGS. 5, 6) may be translated distally relative to snare 122 to cause snare loop 126 to constrict around IMD 110. Snare 122 may constrict around any portion of IMD 110 (e.g., around either IMD distal portion 116 or IMD proximal portion 114). In examples, when snare 122 constricts around a portion of IMD 110 distal to IMD retrieval structure 111, snare 122 may proximally translate (e.g., be proximally translated by a clinician) relative to IMD 110 such that snare loop 126 constricts around IMD retrieval structure 111. In examples, IMD retrieval structure 111 defines a stem 182 (“IMD stem 182”) and a crown 184 (“IMD crown 184”) proximal to IMD stem 182. IMD crown 184 may be configured to substantially cease a proximal translation of snare 122 when snare loop 126 constricts around IMD stem 182, such that snare 122 remains engaged with IMD 110.

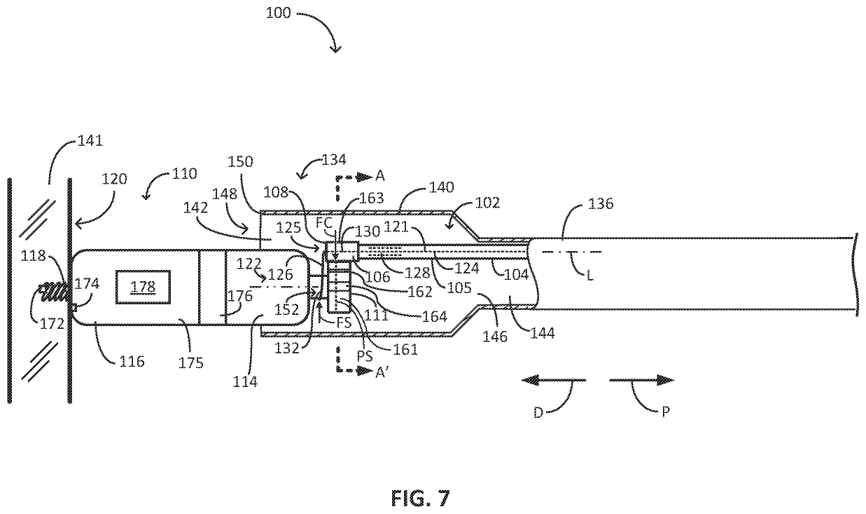

IMD 110 (e.g., IMD retrieval structure 111) may define one or more device recesses such as device recess 162 and device recess 173, and may define one or more device slots such as device slot 164 and device slot 177. A protrusion of head section 106 (e.g., protrusion 160, 166) may configured to insert into device recess 162, 173 and/or device slot 164, 177. In examples, snare 122 is configured to cause head section 106 to exert a contact force (e.g., contact force FC (FIG. 7)) against IMD 110 (e.g., IMP retrieval structure 111) to substantially maintain the protrusion inserted in device recess 162, 173 or device slot 164, 177. Driver 102 is configured such that a torque imparted on driver body 104 (e.g., by a clinician) causes head section 106 to transfer the torque to IMP 110 when the protrusion is inserted in device recess 162, 173 or device slot 164, 173.

Medical system 100 may be configured such that a torque on head section 106 causes head section 106 to substantially roll and/or slip around a perimeter defined by IMD 110 (e.g., crown perimeter P2) to cause the protrusion 160, 166 to insert within device recess 162, 173 or device slot 164, 177. In examples, device recess 162, 173 is configured to substantially capture (e.g., to catch) protrusion 160, 166 as device head 106 substantially rolls and/or slips around the perimeter, such that device head 106 substantially walks around the perimeter toward device slot 165, 177. Device head 106 may be caused to walk around the perimeter using device recess 162, 173 and other device recesses until device head 106 inserts into device slot 164, 177.

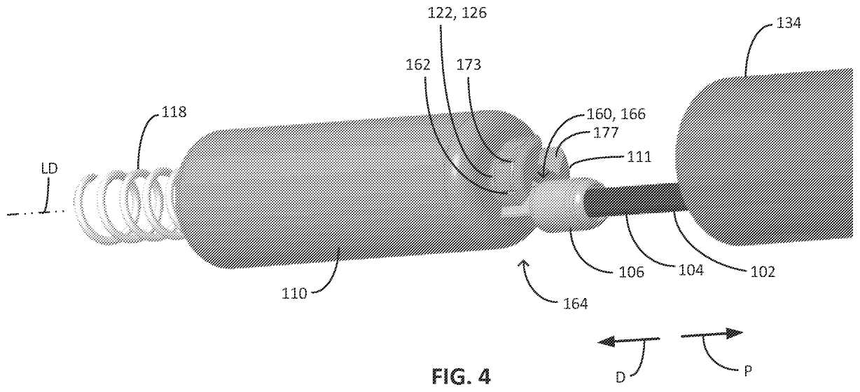

For example, FIG. 4 illustrates head section 106 inserted within device slot 164. Snare 122 exerts a force on IMD stem 182 to cause head section 106 to exert a contact force on IMD crown 184, substantially maintaining head section 106 within device slot 164. With head section 106 substantially maintained within device slot 164, a torque exerted on driver body 1004 (e.g., by a clinician) may be imparted to IMD 110 (e.g., IMD crown 184) by head section 106.

FIG. 5 is a schematic illustration of medical system 100 including snare 122, driver 102, and delivery catheter 134. Snare 122 includes snare body 124 and snare loop 126. Driver 102 includes head section 106 and defines driver lumen 128. Driver lumen 128 defines longitudinal axis L. FIG. 6 illustrates medical system 100 with a cross-section view of driver 102 and delivery catheter 134, with the cross-section cutting plane taken through longitudinal axis L. FIG. 7 is a schematic illustration of medical system 100 with head section 106 engaging IMD 110 (e.g., IMD retrieval structure 111) within delivery receptacle volume 142. Delivery receptacle 140 of delivery catheter 134 is illustrated as a cross-section for clarity, with the cross-section cutting plane taken through longitudinal axis L. Attachment member 118 is engaged with a tissue wall 141 such that IMD 110 remains anchored to tissues within or in proximity to target site 120.

Snare 122 may be configured to translate within driver lumen 128 both distally (e.g., in the distal direction D) and/or proximally (e.g., in the proximal direction P) relative to driver 102 and delivery catheter 134. Snare 122 may extend through driver lumen opening 125 to translate within driver lumen 128. Driver 102 may be configured to translate within delivery catheter lumen 144 both distally and/or proximally relative to snare 122 and delivery catheter 134. Delivery catheter 134 may be configured to translate both distally and/or proximally relative to snare 122 and driver 102. Snare 122 may extend through driver lumen opening 125 to translate within driver lumen 128.

Snare 122 is configured to engage IMD retrieval structure 111 when snare loop 126 is positioned around or in proximity to IMD retrieval structure 111. For example, snare loop 126 may define a loop aperture 152 configured to receive (e.g., substantially surround) at least some portion of IMD retrieval structure 111 when snare loop 126 is positioned around or in proximity to IMD retrieval structure 111. Snare 122 may be configured to cause snare loop 126 (e.g., loop aperture 152) to substantially constrict around IMD retrieval structure when snare loop 126 is engaged with IMD retrieval structure 111. For example, snare 122 may be configured such that, when snare loop 126 engages IMD retrieval structure 111, a proximal force (e.g., a force in the proximal direction P) exerted on snare body 124 causes snare loop 126 (e.g., loop aperture 152) to constrict around IMD retrieval structure 111.

In some examples, snare 122 includes a sheath 154 (“snare sheath 154”) defining a lumen 156 (“sheath lumen 156”). Snare sheath 154 may be configured to slidably translate within driver lumen 128. In FIG. 6, snare sheath 154 is shown as transparent within driver lumen 128 for clarity. Snare body 124 and/or snare loop 126 may be configured to slidably translate within sheath lumen 156, such that a clinician may cause movement of snare sheath 154 (e.g., distal and/or proximal relative movement) relative to snare body 124 and/or snare loop 126. For example, a clinician may cause snare sheath 154 to translate distally relative to driver body 104 when snare loop 126 is engaged with IMD retrieval structure 111 to cause snare loop 126 to constrict around IMD retrieval structure 111. Translation of snare sheath 154 toward snare loop 126 may cause some portion of snare loop 126 to enter sheath lumen 156, at least partially collapsing loop aperture 152 and constricting snare loop 126 around retrieval structure 111.

Snare sheath 154 may be configured to substantially guide driver 102 toward IMD retrieval structure 111 subsequent to causing the constriction of snare loop 126 around IMD retrieval structure 111. Snare sheath 154 may include a distal portion 157 (“snare sheath distal portion 157” (FIG. 6)) configured to be intracorporeal to the patient (e.g., via driver lumen 128) and a proximal portion (“snare sheath proximal portion” (not shown)) which may be extracorporeal to the patient when snare sheath distal portion 157 is intracorporeal. Medical system 100 may be configured such that a clinician may cause relative movement between snare sheath 154 and snare body 124 and/or snare loop 126 using snare proximal portion 123 and/or the snare sheath proximal portion.

Snare body 124 and/or snare loop 126 may translate (e.g., within driver lumen 128 and/or sheath lumen 156) in the distal direction D and/or the proximal direction P relative to driver 102, delivery catheter 134, and/or IMD 110. Snare body 124 may be configured such that a force exerted on snare body 124 (e.g., exerted on snare proximal portion 123, by a clinician) causes a translation of snare body 124 within driver lumen 128 and/or sheath lumen 156, and the translation of snare body 124 causes a translation of snare loop 126. Snare body 124 may be configured to alter a position of snare 122 relative to driver 102, delivery catheter 134, and/or IMD 110. For example, snare 122 may be translated (e.g., by a clinician exerting a force on snare body 124) in the distal direction D substantially toward IMD 110 to place snare loop 126 in proximity to IMD retrieval structure 111. Some portion of snare 122 (e.g., snare body 124) may be translated (e.g., by a clinician exerting a force on snare body 124) in the proximal direction P substantially away from IMD 110 to cause snare loop 126 to constrict around IMD retrieval structure 111, and/or snare sheath 154 may be translated (e.g., by the clinician) distally relative to snare 122 to cause snare loop 126 to constrict around IMD retrieval structure 111. Snare 122 may be configured to translate within driver lumen 128 and/or sheath lumen 156 such that snare loop 126 positions either distal to or proximal to delivery receptacle opening 148. For example, snare loop 126 may position distal to delivery receptacle opening 148 (as depicted in FIGS. 5, 6) and/or may position proximal to delivery receptacle opening 148 (as depicted in FIG. 7).

Driver body 104 is configured to translate within delivery catheter lumen 144 to cause head section 106 to engage IMD 110 (e.g., IMD retrieval structure 111). Driver body 104 may translate (e.g., within delivery catheter lumen 144) in the distal direction D and/or the proximal direction P relative to snare 122, delivery catheter 134, and/or IMD 110. Driver body 104 may be configured such that a force exerted on driver body 104 (e.g., exerted on driver body proximal portion 107, by a clinician) causes a translation of driver body 104 within delivery catheter lumen 144, and the translation of driver body 104 causes a translation of head section 106. Driver body 104 may be configured to alter a position of head section 106 relative to snare 122, delivery catheter 134, and/or IMD 110. For example, head section 106 may be moved (e.g., by a clinician exerting a force on driver body 104) in the distal direction D substantially toward IMD 110 to cause head section 106 to engage IMD retrieval structure 111. Head section 106 may be moved (e.g., by a clinician exerting a force on driver body 104) in the proximal direction P substantially away from IMD 110 to cause head section 106 to disengage from IMD retrieval structure 111. Driver body 104 may be configured to translate within delivery catheter lumen 144 such that head section 106 and/or other portions of driver body 104 position distal to and/or proximal to delivery receptacle opening 148. For example, head section 106 may position distal to delivery receptacle opening 148 (as depicted in FIGS. 5, 6) and/or may position proximal to delivery receptacle opening 148 (as depicted in FIG. 7).

FIG. 7 illustrates head section 106 engaging IMD 110. Snare 122 configured to cause head section 106 to exert a contact force FC on IMD 110 when head section 106 engages IMD 110 and snare 122 exerts a force on IMD 110. For example, in FIG. 7, head section 106 is positioned substantially between first snare portion 130 and IMD retrieval structure 111. Snare 122 engages and exerts a snare-imparted force FS on IMD retrieval structure 111 using second snare portion 132. First snare portion 130 and second snare portion 132 are portions of snare loop 126 and/or snare body 124. Snare 122 and/or head section 106 are configured such that when second snare portion 132 exerts snare-imparted force FS on IMD retrieval structure 111, first snare portion 130 substantially places head section 106 in compression between first snare portion 130 and IMD retrieval structure 111, causing head section 106 to exert contact force FC (e.g., a reaction force) against IMD retrieval structure 111.

Second snare portion 132 may be configured to exert snare-imparted force FS on IMD retrieval structure 111 when a tension is applied to the snare (e.g., when a proximal force is exerted (e.g., by a clinician) on snare distal portion 121). In examples, snare loop 126 surrounds device axis LD of IMD 110 when second snare portion 132 exerts snare-imparted force FS on IMD retrieval structure 111. Head section 106 may be configured such that contact force FC substantially causes head section 106 to maintain contact with IMD retrieval structure 111. In examples, head section 106 is configured to exert contact force FC against a perimeter defined by IMD 110.

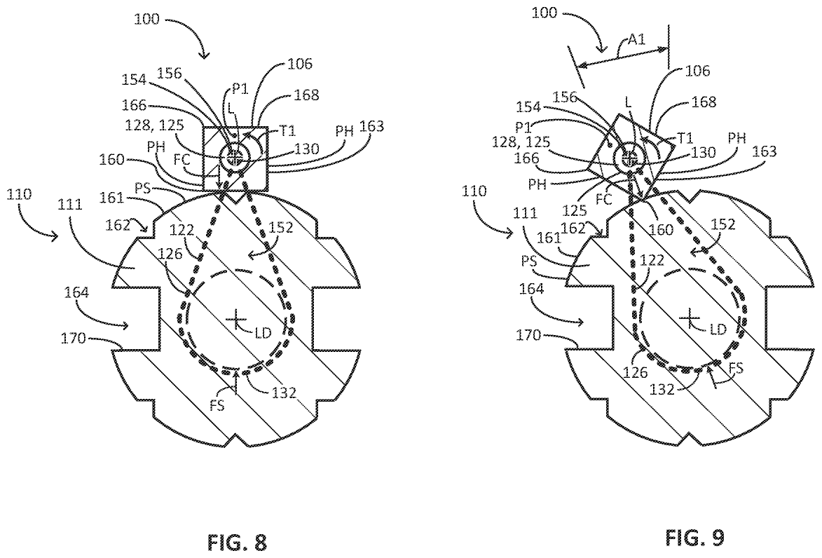

For example, FIG. 8 illustrates a cross-sectional view of head section 106 engaged with IMD retrieval structure 111, taken with a cutting plane indicated as A-A′ in FIG. 7. Snare loop 126 surrounds device axis LD and some portion of IMD retrieval structure 111 as snare loop 126 and/or snare body 124 extends through driver lumen 128 and driver lumen opening 125. In FIG. 8, snare loop 126 is shown in dashed lines, “behind” the cutting plane A-A′. Snare loop 126, snare body 124, and/or first snare portion 130 may extend through sheath lumen 156 of snare sheath 154.

In FIG. 8, second snare portion 132 exerts snare-imparted force FS on IMD retrieval structure 111. Snare 122 causes head section 106 to exert contact force FC on IMD retrieval structure 111 when second snare portion 132 exerts snare-imparted force FS. In examples, head section 106 exerts contact force FC against a structure perimeter PS defined by IMD 110. Contact force FC substantially causes head section 106 to maintain contact with structure perimeter PS of retrieval structure 111. Structure perimeter PS may be, for example, a perimeter defined by an IMD surface 161 of IMD 110 (e.g., IMD retrieval structure 111) which at least partially surrounds device axis LD. In examples, contact force FC substantially causes a head surface 163 to maintain contact with IMD surface 161 and/or structure perimeter PS. Head surface 163 may at least partially surrounds longitudinal axis L. In examples, head surface 163 defines a head perimeter PH at least partially surrounding longitudinal axis L. In examples, contact force FC substantially causes head perimeter PH to maintain contact with IMD surface 161 and/or structure perimeter PS. Head perimeter PH may be substantially perpendicular to longitudinal axis L. Structure perimeter PS may be substantially perpendicular to device axis LD.

Snare 122 is configured to exert snare-imparted force FS when a force substantially parallel to longitudinal axis L is exerted (e.g., by a clinician) on snare proximal portion 123 (FIG. 1). Driver lumen opening 125 may be configured to substantially redirect the force exerted on snare proximal portion 123, such that the redirected force generates snare-imparted force FS at an angle substantially perpendicular or oblique to longitudinal axis L. In examples, snare-imparted force FS includes a first force component acting on IMD retrieval structure 111 in a first direction substantially perpendicular to longitudinal axis L. Contact force FC may include a second force component acting on IMD retrieval structure 111 in a second direction substantially perpendicular to longitudinal axis L. In examples, the second direction is substantially opposite the first direction.

In examples, head section 106 defines a protrusion 160, such as a corner. In examples, protrusion 160 defines some portion of head perimeter PH. In examples, at least a portion of head perimeter PH defines a polygonal curve, and protrusion 160 defines at least a portion of the polygonal curve. Head perimeter PH may define a polygon such as, for example, a rectangle or other polygon. In some examples, at least a portion of head perimeter PH defines a curved or curvilinear segment, and protrusion 160 defines at least a portion of the curved or curvilinear segment. For example, when the portion of head perimeter PH defines a curved or curvilinear segment, this may mean that the curved or curvilinear segment defines a first point defining a first radial displacement from longitudinal axis L, a second point defining a second radial displacement from longitudinal axis L, and a third point defining a third radial displacement from longitudinal axis L, wherein the second radial displacement is greater than the first radial displacement and the third radial displacement. In examples, the second point is between the first point and the third point.

Head section 106 may be configured to transfer a torque from driver body 104 to IMD 110 when protrusion 160 inserts within a device recess 162 or a device slot 164 defined by IMD 110 (e.g., IMD retrieval structure 111). For example, driver body 104 may be configured to transfer a torque T1 around longitudinal axis L to head section 106 when the torque T1 is imparted (e.g., by a clinician) on driver body 104. In examples, head section 106 is configured to substantially slip and/or roll around structure perimeter PS when driver body 104 imparts the torque T1, such that protrusion 160 may insert into device recess 162 or device slot 164. As used here, when head section 106 substantially slips and/or rolls around structure perimeter PS, this may mean that head section 106 rotates about longitudinal axis L and relative to IMD 110 (e.g., IMD retrieval structure 111) as head section 106 contacts structure perimeter PS. In examples, driver body 104 and head section 106 angularly displace relative to IMD 110 when head section 106 substantially rolls or slips around structure perimeter PS. Snare 122 may be configured to cause head section 106 to exert the contact force FC on structure perimeter PS as driver body 104 imparts torque T1 on head section 106.

For example, FIG. 9 illustrates head section 106 having substantially slipped and/or rolled around structure perimeter PS as a result of driver body 104 imparting torque T1 to head section 106. As a result of torque T1 exerted on head section 106 as snare 12 causes head section 106 to exert contact force FC, head section 106 has rotated about longitudinal axis L and relative to IMP retrieval structure 111, as compared to the relative orientation between head section 106 and IMD retrieval structure 111 in FIG. 8. Head section 106 has substantially slipped and/or rolled around structure perimeter PS such that, in addition to rotating relative to IMD retrieval structure 111, a point P1 defined by head section 106 has angularly displaced relative to IMP 110 over an angle A1 relative to IMP retrieval structure 111. Hence, medical system 100 is configured such that, when head section 106 engages IMD 110 (e.g., IMD retrieval structure 111), a torque T1 exerted on head section 106 may alter a position of head section 106 relative to IMD 110. A clinician may exert the torque T1 on driver body 104 to cause head section 106 to substantially slip and/or roll around structure perimeter PS to alter the position of head section 106 relative to IMD 110. In examples, head section 106 and at least a portion of IMD 110 (e.g., IMD retrieval structure 111) are configured to position within delivery receptacle 140 (e.g., within delivery receptacle volume 142) when head section 106 engages IMD 110, and/or when head section 106 slips and/or rolls around structure perimeter PS.

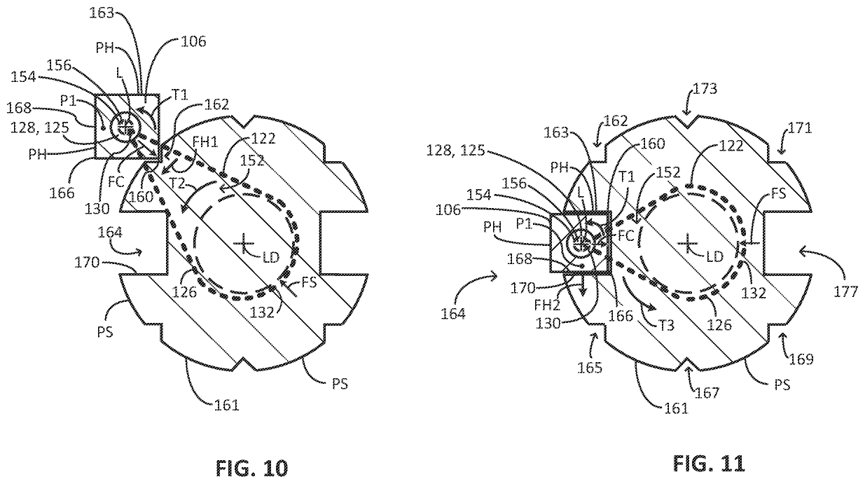

Medical system 100 may be configured such that altering the position of head section 106 relative to IMD 110 (e.g., using torque T1) causes protrusion 160 to insert into device recess 162 or device slot 164. For example, FIG. 10 illustrates head section 106 having substantially slipped and/or rolled around structure perimeter PS such that protrusion 160 is inserted into device recess 162. In examples, medical system 100 (e.g., snare 122) is configured such that the contact FC exerted by head section 106 is transmitted by protrusion 160 to device recess 162 when protrusion 160 inserts within device recess 162. In some examples, medical system 100 is configured such that contact force FC acting on device recess 162 causes protrusion 160 to substantially remain inserted within device recess as torque T1 is applied to head section 106, such that head section 106 transfers at least some portion of the torque T1 to IMD 110 (e.g., IMD retrieval structure 111). The portion of the torque T1 transferred may cause a IMD 110 to revolve substantially about device axis LD.

For example, medical system 100 may be configured such that contact force FC may cause head section 106 and IMD 110 to remain substantially stationary with respect to each other when protrusion 160 inserts into device recess 162 (e.g., such that head section 106 substantially ceases to slip and/or roll over structure perimeter PS). The contact force FC may be increased (e.g., by a clinician) to a level sufficient to cause head section 106 and IMD 110 to remain substantially stationary relative to each other by using snare 122 to increase snare-imparted force FS (thereby increasing contact force FC). When head section 106 and IMD 110 maintain substantially stationary relative positions and torque T1 continues to cause the rotation of head section 106 about longitudinal axis L, longitudinal axis L may substantially orbit around device axis LD of IMD 110 to accommodate the continued rotation of head section 106. With head section 106 and IMD 110 maintaining substantially stationary relative positions, the orbiting of longitudinal axis L around device axis LD may cause protrusion 160 to impart at least some portion of torque T1 to IMD 110, causing rotation of IMD 110 about device axis LD. For example, the orbiting of longitudinal axis L around device axis LD as torque T1 is applied to head section 106 may cause protrusion 160 to impart a torque T2 around device axis LD to IMD 110 (e.g., IMD retrieval structure 111). For example, torque T1 may cause protrusion 160 to exert a head-transmitted force FH1 to device recess 162 to impart the torque T2 to IMD 110. In examples, head-transmitted force FH1 is substantially perpendicular to longitudinal axis L and/or device axis LD.

In similar fashion, medical system 100 may be configured such that altering the position of head section 106 relative to IMD 110 causes protrusion 160 to insert into device slot 164. For example, FIG. 11 illustrates head section 106 having substantially slipped and/or rolled around structure perimeter PS such that protrusion 160 is inserted into device slot 164. Medical system 100 may be configured such that the contact force FC exerted by head section 106 is transmitted by protrusion 160 and/or other portions of head section 106 to device slot 164 when protrusion 160 inserts within device slot 164. Head section 106 may transfer at least some portion of the torque T1 to IMD 110 (e.g., IMD retrieval structure 111) to cause IMD 110 to revolve substantially about device axis LD.

For example, medical system 100 may be configured such that contact force FC may cause head section 106 and IMD 110 to maintain substantially stationary relative positions when protrusion 160 inserts into device slot 164. When head section 106 and IMD 110 maintain substantially stationary relative positions and torque T1 causes longitudinal axis L to substantially orbit around device axis LD to accommodate a continued rotation head section 106, the orbiting of longitudinal axis L around device axis LD as torque T1 is applied to head section 106 may cause protrusion 160 and/or other portions of head section 106 to impart a torque T3 around device axis LD to IMD 110 (e.g., IMD retrieval structure 111). For example, torque T1 may cause protrusion 160 and/or other portions of head section 106 to exert a head-transmitted force FH2 to device slot 164 to impart the torque T3 to IMD 110. In examples, head-transmitted force FH2 is substantially perpendicular to longitudinal axis L and/or device axis LD.

IMD 110 (e.g., IMD retrieval structure 111) may define either one of device recess 162 or device slot 164, or may define both of device recess 162 and device slot 164, to cause protrusion 160 to transfer a torque to IMD 110. In examples, device recess 162 is configured to cause head section 106 to roll around structure perimeter PS (e.g., to substantially cease slipping around structure perimeter PS) when protrusion 160 inserts into device recess 162. The rolling caused by device recess 162 may substantially cause head section 106 to establish an orientation more amenable to insertion with device slot 164. For example, as head section 106 slips and/or rolls around structure perimeter PS, device recess 162 may substantially capture protrusion 160 when protrusion 160 inserts into device recess 162, such that torque T1 causes head section 106 to substantially pivot around protrusion 160 as torque T1 causes head section 106 to revolve about longitudinal axis L. The pivoting of head section 106 around protrusion 160 may re-orient head section 106 relative to IMD 110 (e.g., IMD retrieval structure 111) to an orientation more amenable to insertion with device slot 164. Head section 106 may substantially pivot around protrusion 160 until the revolution of head section 106 about longitudinal axis L causes protrusion 160 to exit device recess 162.

In examples, device recess 162 defines a first portion of structure perimeter PS (“first PS portion”). Device slot 164 may define a second portion of structure perimeter PS (“second PS portion”). In examples, the first PS portion and/or the second PS portion defines a polygonal curve, and device recess 162 and/or device slot 164 defines at least a portion of the polygonal curve. In some examples, the first PS portion and/or the second PS portion defines a curved or curvilinear segment, and device recess 162 and/or device slot 164 defines at least a portion of the curved or curvilinear segment. For example, when the first PS portion and/or the second PS portion defines a curved or curvilinear segment, this may mean that the curved or curvilinear segment defines a primary point defining a primary radial displacement from device axis LD, a secondary point defining a secondary radial displacement from device axis LD, and a tertiary point defining a tertiary radial displacement from longitudinal axis L, wherein the secondary radial displacement is less than the primary radial displacement and the tertiary radial displacement. In examples, the secondary point is between the primary point and the tertiary point.

In some examples, head section 106 may further define at least a second protrusion 166. Second protrusion may be configured similarly to protrusion 160. In examples, a first portion of structure perimeter PS defines protrusion 160 and a second portion of structure perimeter PPS defines second protrusion 166. In examples, device slot 164 is configured to receive both protrusion 160 and at least second protrusion 166 when head section 106 inserts within device slot 164. Device slot 164 may be configured to receive both protrusion 160 and second protrusion 166 when head section 106 imparts the torque T3 to IMD 110. In examples, device slot 164 is configured to receive both protrusion 160 and second protrusion 166 when portions of head section 106 exert head-transmitted force FH2 to device slot 164.

In some examples, head section 106 defines a head bearing surface 168 configured to transfer head-transmitted force FH2 to a slot bearing surface 170 defined by device slot 164. Head bearing surface 168 may be a portion of head surface 163. Slot bearing surface 170 may be a portion of IMD surface 161. Head section 106 may be configured such that head bearing surface 168 contacts slot bearing surface 170 when head section 106 transfers head-transmitted force FH2 to IMD 110. In examples, head section 106 is configured such that head bearing surface 168 is substantially parallel to slot bearing surface 170 when device slot 164 receives protrusion 160 and second protrusion 166. In examples, head section 106 is configured such that head-transmitted force FH2 acts in a direction substantially normal to head bearing surface 168 and/or slot bearing surface 170, such that, for example, the orientation of head-transmitted force FH2 reduces and/or minimizes a likelihood head-transmitted force FH2 causing head section 106 to exit device slot 164.

In examples, device slot 164 is configured to receive a substantially larger portion of head section 106 than device recess 162. For example, device recess 162 may be configured to receive a first portion of head section 106 when protrusion 160 inserts into device recess 162. Device slot 164 may be configured to receive a second portion of head section 106 when protrusion 160 and second protrusion 166 insert in device slot 164. The second portion of head section 106 may be larger (e.g., larger volumetrically) than the first portion of head section 106. In examples, the first PS portion defined by device recess 162 defines a first segment length and the second PS portion defined by device slot 164 defines a second segment length, and the second segment length is greater than the first segment length. In some examples, head section 106 and/or device recess 162 are configured such that head section 106 defines a first maximum radial displacement from device axis LD when head section 106 is inserted in device recess 162, and head section 106 and/or device slot 164 are configured such that head section 106 defines a second maximum radial displacement from device axis LD when head section 106 is inserted in device slot 164, and the first maximum radial displacement is greater than the second maximum radial displacement. In examples, device recess 162 defines a first minimum radial displacement from device axis LD and device slot 164 defines a second minimum radial displacement from device axis LD, and the first minimum radial displacement is greater than the second minimum radial displacement. The second maximum radial displacement, the first maximum radial displacement, the first minimum radial displacement, and/or the second minimum radial displacement may be substantially perpendicular to device axis LD and/or longitudinal axis L.

IMD 110 (e.g., IMD retrieval structure 111) may define a plurality of device recess, such as at least one of device recess 165, device recess 167, device recess 169, device recess 171, and/or device recess 173 (FIG. 11) in addition to device recess 162. Device recess 165, 167, 169, 171, 173 may be an example of and may be configured similarly to device recess 162. IMD 110 (e.g., IMD retrieval structure 111) may define a plurality of device slots, such as device slot 177 (FIG. 11) or another device slot. Device slot 177 may be an example of and may be configured similarly to device slot 164. In examples, IMD 110 defines device slot 164 and device slot 177 on structure perimeter PS and defines one or more of device recess 162, 165, 167, 169, 171, 173 between device slot 164 and device slot 177 on structure perimeter PS. In examples, IMD 110 defines two or more of device recess 162, 165, 167, 169, 171, 173 between device slot 164 and device slot 177 on structure perimeter PS.

In some examples, head surface 163 may define a substantially smooth surface configured to deform and substantially define a protrusion when head section 106 exerts contact force FC on device recess 162, 165, 167, 169, 171, 173. Head section 106 may be configured such that contact force FC causes the deformation. For example, head section 106 may comprise a material (e.g., a relatively soft polymer) which defines head surface 163 and deforms under contact force FC. The material may be, for example, a TPE (ThermoPlastic Elastomer) or LSR (Liquid Silicone Rubber). In examples, the material has a Shore A Hardness greater than or equal to about 10 and less than or equal to about 100. When snare 122 causes head section to exert contact force FC on IMD 110 (e.g., on device recess 162, 165, 167, 169, 171, 173), contact force FC may cause the relatively soft material of head section 106 to deform (e.g., define a protrusion within device recess 162, 165, 167, 169, 171, 173) and substantially grip device recesses 162, 165, 167, 169, 171, 173. Head section 106 may transfer torque to recess 162, 162,165, 167, 169, 171, 173 using the protrusion caused by the deformation of the material, such that head section transfer torque to IMD 110 (e.g., IMD retrieval structure 111).

In some examples, the material defining head surface 163 is configured to produce a frictional force between head surface 163 and IMD surface 161 when contact force FC causes the material of head section 106 to deform. In examples, the friction force may be sufficient to cause head surface 163 to transfer torque to IMD surface 161 without insertion of head section 106 within device recess 162, 165, 167, 169, 171, 173 or device slot 163, 177. In example where the frictional force is sufficient to cause head surface 163 to transfer torque to IMD surface 161 without insertion of head section 106 within device recess 162, 165, 167, 169, 171, 173 or device slot 163, 177, IMD 110 may or may not defines one or more of device recess 162, 165, 167, 169, 171, 173 and/or device slot 163, 177.

Hence, medical system 100 may be configured to transfer torque T1 from driver body 104 to head section 106. Head section 106 may transfer at least a portion of torque T1 to IMD 110 to cause revolution of IMD 110 substantially about device axis LD when, for example, protrusion 160 inserts into device recess 162 and/or protrusion 160 and second protrusion 166 insert into device slot 164. Medical system 100 may be configured such that a clinician may impart the torque T1 on driver body proximal portion 107 to cause the revolution of IMD 110 to implant IMD 110 within an anatomical volume, retrieve IMD 110 from an anatomical volume, and/or otherwise re-orient IMD 110 when IMD 110 is positioned within an anatomical volume.

Referring to, for example, FIGS. 5-7, IMD 110, which in some examples can comprise a pacemaker such as a leadless and/or wholly intracardiac pacemaker, may include one or more electrodes such as electrode 172 supported by attachment member 118, electrode 174 supported by a housing 175 of IMD 110 (“IMD housing 175”), and/or electrode 176 (e.g., a return electrode) supported by IMD housing 175. One or more of electrodes 172, 174, 176 may be electrically connected to operating circuitry 178. Operating circuitry 178 may be configured to deliver therapy to a patient and/or sense physiological signals of the patient using electrodes 172, 174, 176. In examples, at least a portion of operating circuitry 178 is supported by IMD housing 175. In some examples, at least a portion of operating circuitry 178 is supported by another device displaced from IMD 110, such as another device within the patient and/or another device extracorporeal to the patient.

Driver body 104 may be configured to bend and/or define curvatures (e.g., within vasculature of a patient) as well as transfer torque from head section 106 to IMD 110. In examples, driver body 104 defines a flexible portion supporting and/or contiguous with head section 106. The flexible portion may be, for example, a portion of driver body 104 proximal to head section 106 (e.g., one or more portions of driver body distal portion 105 and/or driver body proximal portion 107). In examples, head section 106 defines a first stiffness and the flexible portion defines a second stiffness, wherein the first stiffness is greater than the second stiffness. The first stiffness may be indicative of an extent to which head section 106 resists deformation in response to a force (e.g., a compression force exerted by first snare portion 130) exerted on head section 106. The second stiffness may be indicative of an extent to which the flexible portion resists deformation in response to the force. Hence, the flexible portion (having the lower stiffness) may be configured to bend and/or define curvatures while head section 106 (having the higher stiffness) transfers torque from head section 106 to IMD 110.

Some portion of or substantially all of snare loop 126 may be configured to slidably translate within driver lumen 128 (e.g., via sheath lumen 156) when snare body 124 translates within driver lumen 128 (e.g., via sheath lumen 156). For example, snare loop 126 may be configured to at least partially collapse to slidably translate within driver lumen 128. In examples, snare body 124 is sufficiently flexible to define a curved and/or curvilinear shape within driver lumen 128. In some examples, snare body 124 may be sufficiently rigid to cause snare loop 126 to extend distally beyond driver lumen opening 125 to engage with (e.g., capture) IMD 110. Snare loop 126 and/or snare body 124 may be resiliently biased such that snare loop 126 substantially establishes a particular orientation relative to snare body 124 when snare loop 126 is unconstrained by driver lumen 128. For example, snare loop 126 and/or snare body 124 may be resiliently biased such that snare loop 126 and snare body 124 define an angle (e.g., an angle of about 90 degrees, about 45 degrees, or some other angle) when snare loop 126 is unconstrained by driver lumen 128.

In some examples, instead of or in addition to the protrusion, medical system 100 is configured such that snare 122 frictionally engages IMD 110 (e.g., IMD retrieval structure 111) when snare loop 126 constricts around IMD 110 and driver body 104 causes snare 122 to rotate around device axis LD. The frictional engagement of snare loop 126 and IMD 110 as snare 122 rotates around device axis LD may impart a torque on IMD 110, causing IMD 110 to revolve about device axis LD. In examples, snare 122 defines a textured surface (e.g., a surface defining a texture such as a surface roughness and/or surface undulations) configured to increase the frictional engagement with IMD 110. In examples, IMD 110 (e.g., IMD retrieval structure 111) defines one or more structure protrusions configured to increase the frictional engagement with IMD 110 and/or enhance a force transfer from snare loop 126 to IMD 110.

For example, FIG. 12 illustrates a portion of driver 102 and IMD 110 with driver 102 and snare 122 proximal to IMD 110. Snare 122 extends through driver lumen 128 (e.g., via sheath lumen 156) with snare loop 126 extended distal to driver lumen opening 125. FIG. 13 illustrates a cross-sectional view of a cross-sectional view of IMD 110 taken with a cutting plane indicated as B-B′ in FIG. 12, and with driver 102 and snare 122 proximal to IMD 110. In FIG. 13, the distal direction D proceeds out of the page while the proximal direction P proceeds into the page.

Snare loop 126 includes a snare surface 180 configured to contact IMD 110 (e.g., IMD retrieval structure 111) when snare loop 126 surrounds device axis LD and constricts around IMD 110. Snare surface 180 defines a textured surface. The textured surface may define, for example, a surface roughness or an undulating surface. The surface roughness may be characterized by, for examples, a profile roughness parameter (e.g., Ra, Rz, Rq, or another roughness parameter) indicative of a deviation from an ideal surface. Snare surface 180 may be configured such that when snare loop 126 tightens around IMD 110, the surface roughness enhances and/or increases the frictional force between snare surface 180 and IMD 110 when snare 122 causes snare surface 180 to impart a snare-imparted force on IMD 110. The surface undulations may be characterized by an surface of snare loop 126 configured to exhibit a sinuous shape, wavelike shape, or other shape wherein a mean surface level defines a varying radial displacement from an snare axis extending within a body of the snare loop. For examples, the surface undulations may be defined by a coil defining snare loop 126 and surrounding the snare axis, a plurality of ridges and/or depressions defined on snare surface 180, or other surface features configured to define the varying radial displacement from the snare axis.

Snare 122 is configured to cause snare surface 180 to contact IMD 110 (e.g., IMD retrieval structure 111) when snare 122 engages IMD 110. In examples, IMD retrieval structure 111 defines a stem 182 (“IMD stem 182”) supported by IMD proximal portion 114 and a crown 184 (“IMD crown”) supported by IMD stem 182. IMD crown 184 may be configured to limit and/or eliminate a tendency of snare 122 to slip off of IMD retrieval structure 111 when snare loop 126 constricts around IMD stem 182 and a proximal force is exerted (e.g., by a clinician) on snare 122 (e.g., snare body 124) (e.g., when a tension is applied to snare 122). IMD crown 184 may be configured such that, when the snare loop 126 constricts around IMD stem 182 and the proximal force is exerted (e.g., when the tension is applied), IMD crown 184 contacts snare loop 126 to substantially cease a translation of snare loop 126 in the proximal direction. IMD crown 184 may be configured such that, when IMD crown 184 contacts snare loop 126 and the proximal force is exerted (e.g., when the tension is applied), the contact between snare loop 126 and IMD crown 184 causes snare 122 to exert a proximal force on IMD 110 (e.g., on IMD retrieval structure 111).

In examples, IMD stem 182 defines a stem surface 186 at least partially surrounding device axis LD. IMD crown 184 may define a crown surface 188 at least partially surrounding device axis LD. Stem surface 186 and/or crown surface 188 may be a portion of IMD surface 161 (FIGS. 5-11). In examples, crown surface 188 defines a greater radial extension from device axis LD than stem surface 186. For example, stem surface 186 may define a stem radial displacement from device axis LD to stem surface 186 and crown surface 188 may define a crown radial displacement from device axis LD. The crown radial displacement may be greater than the stem radial displacement. In examples, the crown radial displacement and/or the stem radial displacement are substantially perpendicular to device axis LD. In examples, stem surface 186 defines a stem perimeter P1 extending at least partially around device axis LD. Crown surface 188 may define a crown perimeter P2 extending at least partially around device axis LD. In examples, stem perimeter P1 and/or crown perimeter P2 are substantially perpendicular to device axis LD. Stem perimeter P1 and/or crown perimeter P2 may be examples of structure perimeter PS (FIGS. 5-11).

As before, snare 122 may translate (e.g., via driver lumen 128 and/or sheath lumen 156) distally from driver distal end 108 such that snare loop 126 surrounds some portion of IMD 110 (e.g., IMD retrieval structure 111). Snare 122 may receive the portion of IMD 110 within loop aperture 152 when snare 122 translates distally from driver distal end 108. Snare sheath 154 may slidably translate distally within driver lumen 128 relative to snare body 124 and/or snare loop 126 to cause snare loop 126 to constrict around IMD 110. In addition to or instead of the translation of snare sheath 154, driver body 104 and head section 106 may translate distally relative to snare body 124 and/or snare loop 126 to cause snare loop 126 to constrict around IMD 110. Driver body 104 and head section 106 may translate substantially over snare 122 as snare 122 engages IMD 110, such that snare 122 substantially guides head section 106 to a position between first snare portion 130 and IMD 110 (e.g., the IMD crown 184).

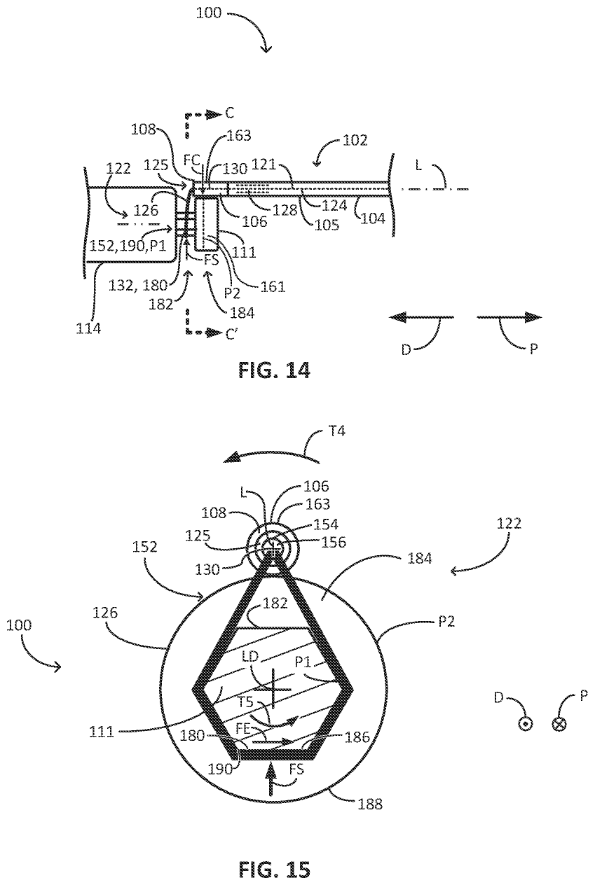

For example, FIG. 14 illustrates snare loop 126 constricting around IMD stem 182 as head section 106 engages IMD crown 184. FIG. 15 illustrates a cross-sectional view of a cross-sectional view of IMD 110 taken with a cutting plane indicated as C-C′ in FIG. 14, and with head section 106 engaging IMD crown 184. In FIG. 15, the distal direction D proceeds out of the page while the proximal direction P proceeds into the page.

Snare 122 extends through driver lumen opening 125 as snare loop 126 (e.g., second snare portion 132) snare engages IMD stem 182 and exerts the snare-imparted force FS on ID stem 182. The snare-imparted force FS exerted by second snare portion 132 causes first snare portion 130 to substantially press head section 106 against IMD crown 184, causing head section 106 to exert contact force FC on IMD crown 184. Driver lumen opening 125 may substantially redirect a force exerted on snare 122 (e.g., by a clinician) from a first direction substantially parallel to longitudinal axis L to a second direction defining an oblique angle to longitudinal axis L, such that the redirected force causes second snare portion to exert snare-imparted force FS on IMD stem 182 at a direction oblique to longitudinal axis L. Snare-imparted force FS may substantially pull IMD crown 184 into head section 106 such that head section 106 exerts contact force FC on IMD crown 184.

Snare 122 (e.g., first snare portion 130) may substantially place head section 106 in compression between first snare portion 130 and IMD crown 184 when snare 122 substantially pulls IMD crown 184 into head section 106. Head section 106 may be configured to exert a first reaction force on first snare portion 132 in response to the compression (e.g., a reaction force substantially equal and opposite to contact force FC). Likewise, when second snare portion 132 exerts snare-imparted force FS on IMD stem 182, snare 122 may be configured such that IMD stem 182 exerts a second reaction force on second snare portion 132. The first reaction force and the second reaction force may act in substantially opposite directions, such that the first reaction force and the second reaction force place snare 122 (e.g., snare loop 126) in tension.

Snare surface 180 may be configured to contact stem surface 186 when snare loop 126 constricts around IMD stem 182. Snare surface 180 may be configured such that, when snare loop 126 constricts around stem surface 186, the textured surface of snare surface 180 causes a frictional force between snare surface 180 and stem surface 186. The magnitude of the frictional force may be dependent on the magnitude of snare-imparted force FS. In examples, in addition to the frictional force, surface undulations of snare surface 180 may substantially bear against stem surface 186, causing transmission of forces from snare 122 to IMD stem 182. The degree to which the surface undulations transmit forces to IMD stem 182 may be dependent on the magnitude of snare-imparted force FS. In examples, the frictional force and/or bearing forces imparted to stem surface 186 by snare surface 180 are sufficient to cause snare 122 to substantially grip IMD stem 182, such that a rotation of snare 122 and/or driver body 104 around device axis LD causes IMD 110 to revolve about device axis LD.

For example, snare 122 may be configured such that a torque T4 exerted on snare 122 causes snare 122 to rotate about device axis LD when snare loop 126 constricts around IMD 110 (e.g., IMD stem 182). Under the influence of snare-imparted force FS, snare surface 180 may generate an engagement force FE acting on stem surface 186. In some examples, engagement force FE includes a frictional force (e.g., caused by a surface roughness) imparted on stem surface 186 by snare surface 180. In some examples, engagement force FE includes a force transmitted to stem surface 186 by an undulation of snare surface 180. Engagement force FE may cause snare 122 to substantially grip IMD stem 182, such that snare 122 imparts a torque T5 to IMD 110 (e.g., IMD stem 182). Torque T5 may cause a IMD 110 to revolve about device axis LD. In examples, medical system 100 is configured such that when torque T4 is exerted on snare 122 in a first rotational direction around device axis LD, snare 122 imparts the torque T5 to IMD 110 in the first rotational direction around device axis LD.

Snare 122 snare may be configured such that increasing a proximally-directed force on snare 122 (e.g., increasing the tension applied to snare 122) causes an increase in snare-imparted force FS, such that snare 122 substantially grips IMD 110 (e.g., IMD stem 182). Increasing the proximally-directed force on snare 122 may increase engagement force FE between snare surface 180 and stem surface 186, such that relative movement between snare 122 and IMD 110 (e.g., IMD stem 182) is limited and/or substantially eliminated when snare 122 experiences the torque T4. Hence, medical system 100 may be configured such that a tension applied to snare 122 (e.g., imparted by a clinician) generates a sufficient magnitude of engagement force FE to cause snare 122 to impart torque T5 to IMD 110.

Driver body 104 may be configured to impart the torque T4 to snare 122. For examples, driver body 104 may be configured to impart a driver-imparted force to snare 122 when snare 122 engages IMD 110 (e.g., IMD stem 182) and driver body rotates around device axis LD. Driver body 104 (e.g., an inner lumen wall defining driver lumen 128) may act against snare 122 (e.g., first snare portion 130) to impart the driver-imparted force to snare 122. The driver-imparted force may cause torque T4 on snare 122. In examples, snare 122 (e.g., snare body 124) may be configured to substantially key to driver body 104 to assist driver body 104 in causing torque T4. For example, an inner surface of driver lumen 128 (e.g., an inner surface in proximity to driver lumen opening 125) may define a structure configured to substantially bear against snare 122 when driver body 104 rotates around longitudinal axis L, causing driver body 104 to exerts the force on snare 122. The structure may be, for example, an inner surface recess substantially extending radially away from longitudinal axis L, and/or may be an inner surface protrusion extending radially toward longitudinal axis L. Driver body 104 may impart the force on snare 122 by substantially transmitting the force from the inner surface recess and/or inner surface protrusion to snare 122.

As previously discussed, a torque on driver body 104 (e.g., exerted by a clinician) may cause longitudinal axis L to rotate around (e.g., substantially orbit) device axis LD of IMD 110 when snare 122 engages IMD 110 (e.g., IMD stem 182). The orbiting of longitudinal axis L around device axis LD may cause driver body 104 to impart the driver-imparted force to snare 122, causing torque T4 on snare 122. In some examples, head section 106 may revolve about longitudinal axis L and relative to IMD 110 as longitudinal axis L orbits around device axis LD. In some examples, head section 106 and IMD 110 may remain substantially stationary with respect to each other as longitudinal axis L orbits around device axis LD. For example, contact force FC may cause head section 106 and IMD 110 may remain substantially stationary with respect to each other as longitudinal axis L orbits around device axis LD. In some examples, head section 106 may revolve about longitudinal axis L and relative to snare 122 (e.g., first snare portion 130 and/or other portions of snare 122) as longitudinal axis L orbits around device axis LD.

In examples, IMD stem 182 defines one or more structure protrusions such as structure protrusion 190 (e.g., a corner). Structure protrusion 190 may be configured to increase and/or enhance engagement force FE when snare surface 180 contacts stem surface 186. In examples, structure protrusion 190 defines some portion of stem perimeter P1. In examples, at least a portion of stem perimeter P1 defines a polygonal curve, and structure protrusion 190 defines at least a portion of the polygonal curve. Stem perimeter P1 may define a polygon such as, for example, a hexagon, octagon, or other polygon. In some examples, at least a portion of stem perimeter P1 defines a curved or curvilinear segment, and structure protrusion 190 defines at least a portion of the curved or curvilinear segment. For example, when the portion of structure perimeter P1 defines a curved or curvilinear segment, this may mean that the curved or curvilinear segment defines a first-defined point defining a first-defined radial displacement from longitudinal axis L, a second-defined point defining a second-defined radial displacement from longitudinal axis L, and a third-defined point defining a third-defined radial displacement from longitudinal axis L, wherein the second-defined radial displacement is greater than the first-defined radial displacement and the third-defined radial displacement. In examples, the second-defined point is between the first-defined point and the third-defined point.

Structure protrusion 190 may be configured to substantially bear against some portion of snare surface 180 (e.g., an undulation of snare surface 180) when snare surface 180 rotates around device axis LD. Structure protrusion 190 may be configured such that some portion of snare surface 180 transmits at least a portion of engagement force FE to IMD 110 as a contact force on structure protrusion 190. Structure protrusion 190 may define a bearing surface configured to contact snare surface 180 when snare surface 180 transfers the contact force. In examples, the bearing surface is configured such that the contact force includes one or more force components acting on the bearing surface in a direction substantially non-parallel to the bearing surface. For example, the bearing surface may be configured such that snare surface 180 imparts a contact force having a force component substantially normal to the bearing surface, and/or having a force component acting on the bearing surface in a direction oblique to the bearing surface. In examples, when snare surface 180 exerts the contact force on structure protrusion 190, the bearing surface acts to minimize and/or limit relative movement between snare surface 180 and structure protrusion 190, such that snare 122 substantially grips IMD stem 182. In examples, medical system 100 is configured such that an increase in snare-imparted force FS causes an increase in the contact force transmitted from snare surface 180 to structure protrusion 190. In some examples, an undulation of snare surface 180 is configured to contact the bearing surface of structure protrusion 190 to transfer the contact force.

In examples, IMD stem 182 defines a plurality of structure protrusions arranged substantially circumferentially around device axis LD. For examples, in addition to structure protrusion 190, IMD stem may define structure protrusion 191, structure protrusion 192, structure protrusion 193, structure protrusion 194, and or structure protrusion 195 (FIG. 13). Each of structure protrusions 191, 192, 193, 194, 195 may be configured similarly to structure protrusion 190. For example, each of structure protrusions 191, 192, 193, 194, 195 may define an individual portion of stem perimeter P1. Each of structure protrusions 191, 192, 193, 194, 195 may define an individual polygonal curve defined by stem perimeter P1. Each of structure protrusions 191, 192, 193, 194, 195 may define a curved or curvilinear segment defined by structure perimeter P1. Each of structure protrusions 191, 192, 193, 194, 195 may include an individual bearing surface configured to receive an individual contact force from snare surface 180, such that one or more of structure protrusions 191, 192, 193, 194, 195 may acts to minimize and/or limit relative movement between snare 122 and IMD stem 182. In examples, protrusions 190, 191, 192, 193, 194, 195 are arranged such that each of protrusions 190, 191, 192, 193, 194, 195 extend in direction radially outward from device axis LD.

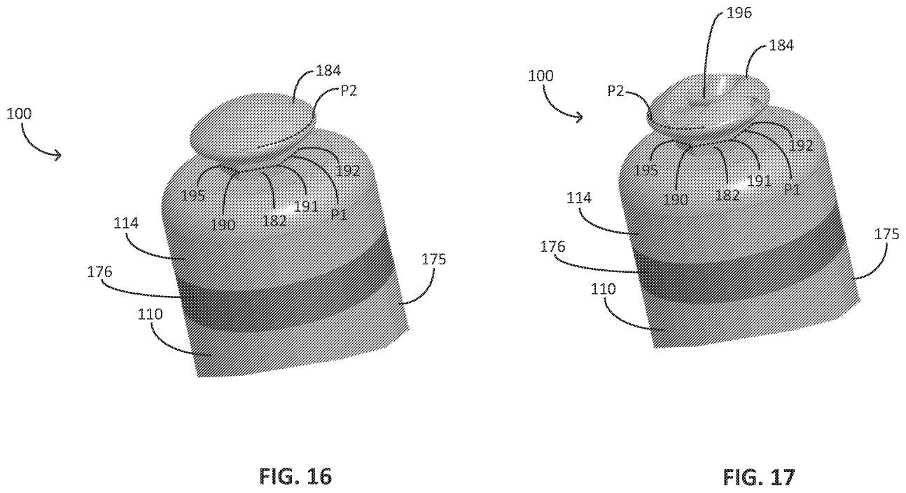

FIG. 16 illustrates a perspective view of an example IMD 110 defining a plurality of structure protrusions arranged circumferentially around device axis LD. FIG. 17 illustrates a perspective view of an example IMD 110 defining a plurality of structure protrusions and including a pin 196 supported by IMD retrieval structure 111 (e.g., IMD crown 184). FIG. 16 and FIG. 17 illustrates structure protrusion 190, structure protrusion 191, structure protrusion 192, and structure protrusion 195 defined around stem perimeter P1. In FIG. 16 and FIG. 17, IMD 110 may include additional structure protrusions (e.g., additional structure protrusions hidden in the views of FIG. 16 and FIG. 17). Snare loop 126 may constrict around IMD stem 182 such that the plurality of protrusions acts to minimize and/or limit relative movement between snare 122 and IMS stem 182, such that snare 122 may transfer a torque to cause revolution of IMD 110 substantially about device axis LD. Pin 196 is configured to transfer a force imparted to pin 196 to at least IMD housing 175, such that medical system 100 and/or another medical system may alter a position of IMD 110 by engaging pin 196.

Referring to, for example, FIGS. 5-7, operating circuitry 178 may include fixed function circuitry and/or programmable operating circuitry. In examples, operating circuitry 178 may include circuitry configured to perform one or more functions of operating circuitry 178, such as therapy delivery circuitry, sensing circuitry, processing circuitry, switching circuitry, communication circuitry, and/or other circuitries. Operating circuitry 178, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, operating circuitry 178 includes multiple components, such as any combination of one or more microprocessors, one or more DSPs, one or more ASICs, or one or more FPGAs, as well as other discrete or integrated logic circuitry, and/or analog circuitry.

Functions attributed to operating circuitry 178 may be embodied as software, firmware, hardware or any combination thereof. Operating circuitry 178 may include, for instance, a variety of capacitors, transformers, switches, and the like configured to perform the functions of operating circuitry 178. In examples, operating circuitry 178 may be configured to communicate with another device, such as a patient input/output device, a clinician input/output device, and/or others. Operating circuitry 178 may include any suitable hardware, firmware, software or any combination thereof for communicating with another device. In addition, operating circuitry 178 may communicate with a networked computing device and a computer network. In examples, operating circuitry 178 and/or other circuitry of medical system 100 is configured to deliver stimulation signals to and/or receive sensing signals from electrodes 172, 174, 176 and/or other electrodes and/or sensors within medical system 100 or external to medical system 100. Operating circuitry 178 may be configured to provide electrical signals, e.g., pacing therapy, to electrodes 172, 174, 176. Operating circuitry 178 may be configured to receive electrical signals, e.g., sensed cardiac electrical signals, from electrodes 172, 174, 176.

Medical system 100 (e.g., operating circuitry 178) can also include memory configured to store program instructions, such as software, which may include one or more program modules, which are executable by operating circuitry 178. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by operating circuitry 178 cause operating circuitry 178 to perform various functions described herein and/or other functions of operating circuitry 178.

IMD housing 175 may enclose operating circuitry 178 and/or other circuitry within medical system 10. IMD housing 175 may be configured to fluidly isolate operating circuitry 178 and/or other circuitry from an environment in contact with an exterior surface of IMD housing 175. In examples, IMD housing 175 is configured to hermetically seal an enclosure defined by IMD 110 and holding operating circuitry 178 and/or other circuitry. IMD housing 175 may be configured to define shapes that are easily accepted by the patient's body while minimizing patient discomfort. For example, IMD housing 175 may define a substantially cylindrical shape with cylindrical sidewalls. In other examples, ID housing 175 may define substantially rectangular or other non-cylindrical shapes. IMD housing 175 may define shapes in which corners and edges are designed with relatively large radii, in order to present a housing having smoothly contoured exterior surfaces. In examples, attachment member 118 is coupled to IMD housing 175.

As used here, when a first portion of a system (e.g., medical system 100) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

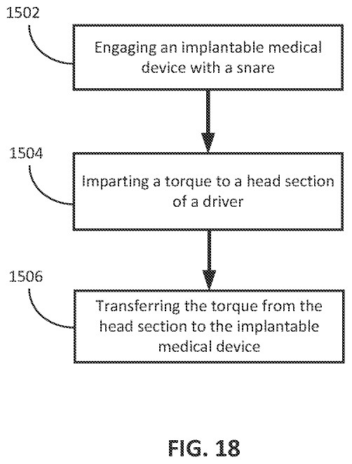

A technique for imparting a torque using a medical system 100 is illustrated in FIG. 18. Although the technique is described mainly with reference to medical system 100 of FIGS. 1-17, the technique may be applied to other medical systems in other examples.

The technique includes engaging an IMD 110 with a snare 122 configured to exert a force on IMD 110 to cause a head section 106 of a driver 102 to exert a contact force FC on IMD 110 (1502). In some examples, a snare 122 engages IMD 110 using a snare loop 126. Snare loop 126 may exert the proximal force on IMD 110 when a proximal force is exerted (e.g., by a clinician) on a snare body 124 supporting snare loop 126. In examples, snare body 124 and/or snare loop 126 extend through a driver lumen 128 and a driver lumen opening 125 defined by a driver body 104 of a driver 102. Snare loop 126 may at least partially collapse (e.g., around IMD 110) when snare body 124 slidably translates within driver lumen 128. In examples, a snare sheath 154 is translated (e.g., translated distally) relative to snare body 124 to constrict snare loop 126 around IMD 110. In examples, head section 106 exerts contact force FC on a retrieval structure 111 of IMD 110. In examples, head section 106 may translate distally along snare 122 to position between a first snare portion 130 and IMD 110.

Snare 122 may substantially compress head section 106 between first snare portion 130 and IMD 110 when a second snare portion 132 exerts the force on IMD 110. Snare 122 may impart a snare-imparted force FS on IMD 110 (IMD retrieval structure 111) when snare 122 exerts the force on IMD 110. Snare 122 may impart snare-imparted force FS using second snare portion 132. When second snare portion 132 exerts snare-imparted force FS on IMD 110, first snare portion 130 substantially places head section 106 in compression between first snare portion 130 and IMD retrieval structure 111, causing head section 106 to exert contact force FC (e.g., a reaction force) against IMD retrieval structure 111.

The technique includes imparting a torque T1, using a driver body 104 of driver 102, on head section 106 (1504). Driver body 104 may impart torque T1 on head section 106 to cause head section 106 to rotate within an anatomical volume (e.g., a heart chamber) defined by a patient. Torque T1 may cause head section 106 to substantially slip and/or roll around a structure perimeter PS defined by IMD 110 (e.g., IMD retrieval structure 111). In examples, head section 106 substantially slips and/or rolls around structure perimeter PS to cause a protrusion 160, 190 to insert into a device recess 162, 163, 165, 167, 171, 173 or a device slot 164, 177 defined by IMD 110.

The technique includes transferring, using head section 106, torque T1 from driver body 104 to IMD 110 (e.g., IMD retrieval structure 111) when snare 122 causes head section 106 to exert contact force FC and protrusion 160, 190 inserts into device recess 162, 163, 165, 167, 171, 173 or device slot 164, 177. Head section 106 may transfer at least one of a head-imparted force FH1 or a head-imparted FH2 to IMD 110 to transfer the torque T1. In examples, protrusion 160, 190 transfers head-imparted force FH1 to device recess 162, 163, 165, 167, 171, 173. In examples, protrusion 160, 190 transfers head-imparted force FH2 to device slot 164, 177.

Head section 106 may cause IMD 110 to rotate about a device axis LD defined by IMD 110 when head section 106 transfers the torque from head section 106 to IMD 110. In examples, head section 106 causes an attachment member 118 of IMD 110 to disengage from tissue within or in proximity to a target site 120 within the patient when head section 106 causes IMD 110 to rotate about a device axis LD. In examples, head section 106 causes attachment member 118 of IMD 110 to engage tissue within or in proximity to target site 120 when head section 106 causes IMD 110 to rotate about device axis LD. In examples, head section 106 causes one or more device components of IMD 110 to rotate about device axis LD when head section 106 causes IMD 110 to rotate about device axis LD.

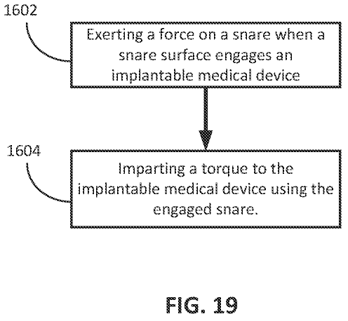

An additional technique for imparting a torque using a medical system 100 is illustrated in FIG. 19. Although the technique is described mainly with reference to medical system 100 of FIGS. 1-17, the technique may be applied to other medical systems in other examples. Further, the technique of FIG. 19 may be used instead of or in addition to the technique of FIG. 18.

The technique includes applying a tension to snare 122 when a snare surface 180 frictionally engages IMD 110 (1602). The technique may include exerting a force on snare 122 to apply the tension. In some examples, snare surface 180 is a portion of snare loop 126. In examples, driver 102 (e.g., head section 106) exerts the force on snare 122. Driver 102 may be positioned between first snare portion 130 and IMD 110. In examples, driver 102 imparts the force on snare 122 by exerting a torque T4 on snare 122 around device axis LD.

The technique includes imparting a torque on IMD 110 by imparting an engagement force FE from snare 122 to IMD 110 when driver 102 exerts the force on the snare (1604). In examples, the technique includes constricting snare loop 126 around IMD 110 by slidably translating snare body 124 within driver lumen 128 and/or translating snare sheath 154. In examples, head section 106 translates distally along snare 122 to position between first snare portion 130 and IMD 110. In examples, snare 122 imparts snare-imparted force FS on IMD 110 using second snare portion 132.

Snare-imparted force FS may cause contact between snare surface 180 and IMD 110 (e.g., stem perimeter P1 and/or crown perimeter P2). Snare surface 180 may include a textured surface which increases engagement force FE when snare-imparted force FS increases. In some examples, the technique includes exerting a force (e.g., by a clinician) on snare 122 to increase snare-imparted force FS to increase engagement force FE.

Snare 122 may substantially surround stem perimeter P1 and/or crown perimeter P2 when snare 122 frictionally engages IMD 110. Stem perimeter P1 and/or crown perimeter P2 may substantially surround device axis LD. In examples, second snare portion 132 substantially surrounds stem perimeter P1 and/or crown perimeter P2 when the snare 122 frictionally engages IMD 110. In examples, IMID retrieval structure 111 defines retrieval structure protrusion 190, 191, 192, 193, 194, 194. Structure protrusion 190, 191, 192, 193, 194, 195 may act to increase and/or enhance engagement force FE when second snare portion 132 transfers torque T4 to IMD 110.

Snare 122 may cause IMD 110 to rotate about a device axis LD defined by IMD 110 when snare 122 transfers torque T4 to IMD 110. In examples, snare 122 causes an attachment member 118 of IMD 110 to disengage from tissue within or in proximity to a target site 120 within the patient when snare 122 causes IMD 110 to rotate about a device axis LD. In examples, snare 122 causes attachment member 118 of IMD 110 to engage tissue within or in proximity to target site 120 when snare 122 causes IMD 110 to rotate about device axis LD. In examples, snare 122 causes one or more device components of IMD 110 to rotate about device axis LD when snare 122 causes IMD 110 to rotate about device axis LD.

The technique of FIG. 18 and/or FIG. 19 may include positioning, using a delivery catheter 134, at least a portion of snare 122, head section 106, and/or driver body 104 within the anatomical volume of the patient. In examples, delivery catheter 134 positions at least a portion of snare 122, head section 106, and/or driver body 104 within a chamber of a heart of the patient. Delivery catheter 134 may transport at least a portion of snare 122, head section 106, and/or driver body 104 through vasculature of the patient. In examples, at least a portion of snare 122, head section 106, and/or driver body 104 positions within a delivery receptacle volume 142 defined by delivery catheter 134. Driver body 104 may cause head section 106 to position within delivery receptacle volume 142 by translating through a delivery catheter lumen 144. Snare 122 may position within delivery receptacle volume by translating through a delivery catheter lumen 144.

The disclosure includes the following examples.

Example 1: A medical system, comprising: a driver configured to impart a torque on an implantable medical device within an anatomical volume defined by a body of a patient, the driver including a head section, wherein the driver comprises a lumen extending through at least a portion head section, the lumen extending to a lumen opening defined by the head section, and wherein the head section or a protrusion thereof is configured to insert within a device recess or a device slot of the implantable medical device; and a snare configured to slidably translate within the lumen and through the lumen opening to engage the implantable medical device, wherein the snare is configured to cause the head section to exert a contact force on the implantable medical device when the snare engages the implantable medical device and imparts a force on the implantable medical device, and wherein the head section is configured to transfer the torque from the driver body to the implantable medical device when the snare causes the contact force and the protrusion inserts within the device recess or the device slot.

Example 2: The medical system of example 1, wherein the lumen defines a longitudinal axis, and wherein the head section defines a head perimeter surrounding the longitudinal axis, and wherein the protrusion defines at least a portion of the head perimeter.

Example 3: The medical system of example 2, wherein the head perimeter is substantially perpendicular to the longitudinal axis.

Example 4: The medical system of any of examples 1-3, wherein the protrusion is configured to transmit the torque to a retrieval structure defined by the implantable medical device.

Example 5: The medical system of any of examples 1-4, wherein the snare is configured to exert the force on the implantable medical device when a proximal force substantially parallel to the longitudinal axis of example 2 is exerted on the snare, and wherein the head section is configured to exert the contact force in a direction substantially perpendicular to the longitudinal axis of example 2.

Example 6: The medical system of any of examples 1-5, wherein the snare is configured to at least one of: cause the head section to increase the contact force when the snare increases the force exerted on the implantable medical device, or cause the head section to decrease the contact force when the snare decreases the force exerted on the implantable medical device.

Example 7: The medical system of any of examples 1-6, wherein when the driver body imparts a torque on the head section and the head section imparts the contact force on the implantable medical device, the head section is configured to at least one of substantially roll or slip around a structure perimeter of the implantable medical device to cause the protrusion to insert within the device recess or the device slot, wherein the head section exhibits a relative rotation with respect to the implantable medical device when the head section substantially rolls or slips around the structure perimeter.

Example 8: The medical system of any of examples 1-7, wherein the longitudinal axis defines one or more line segments, wherein the one or more line segments include at least one of a straight line segment, a curved line segment, or a curvilinear line segment.

Example 9: The medical system of any of examples 1-8, wherein the head section defines a plurality of protrusions, wherein each protrusion is configured to insert within the device recess or the device slot of the implantable medical device, and wherein the head section is configured to transfer the torque from the driver body to the implantable medical device when any one of the plurality of protrusions inserts within the device recess or the device slot.

Example 10: The medical system of any of examples 1-9, wherein the head section is configured to rotate about the longitudinal axis of example 2 when driver body rotates about the longitudinal axis of example 2.

Example 11: The medical system of any of examples 1-10, wherein the snare is configured to cause the head section to exert the contact force as the head section rotates about the longitudinal axis of example 2.

Example 12: The medical system of any of examples 1-11, wherein the protrusion is configured to at least one of: impart a head-transmitted force on the device recess when the protrusion inserts into the device recess and the head section transfers the torque from the driver body to the implantable medical device, or impart the head-transmitted force on the device slot when the protrusion inserts into the device slot and the head section transfers the torque from the driver body to the implantable medical device.

Example 13: The medical system of example 12, wherein the head section is configured to exert the head-transmitted force in a direction substantially perpendicular to the longitudinal axis when the driver delivers the torque in a rotational direction around the longitudinal axis.

Example 14: The medical system of any of examples 1-13, wherein at least a portion of the perimeter defines a polygonal curve, and wherein the protrusion defines at least a portion of the polygonal curve.

Example 15: The medical system of example 14, wherein the perimeter defines a polygon.

Example 16: The medical system of example 14 or example 15, wherein the perimeter defines a rectangle.

Example 17: The medical system of any of examples 1-16, further comprising the implantable medical device, wherein the implantable medical device includes a retrieval structure defining at least one of the device recess or the device slot, wherein the retrieval structure defines a device axis and a structure perimeter surrounding the device axis, and wherein the at least one of device recess or the device slot defines at least a portion of the structure perimeter.

Example 18: The medical system of example 17, wherein the retrieval structure defines a plurality of device recesses, wherein each individual device recess defines an individual portion of the structure perimeter.

Example 19: The medical system of example 17 or example 18, wherein the retrieval structure defines a plurality of slots, wherein each individual slot defines a singular portion of the structure perimeter.

Example 20: The medical system of any of examples 17-19, wherein the retrieval structure defines the device recess and the slot, wherein the slot defines a first minimum radial displacement from the device axis and the device recess defines a second minimum radial displacement from the device axis, and wherein the first minimum radial displacement is less than the second minimum radial displacement.

Example 21: The medical system of any of examples 17-20, wherein the retrieval structure defines the device recess and the slot, wherein the head section defines a first maximum radial displacement from the device axis when the protrusion inserts into the device recess and defines a second maximum radial displacement when the protrusion inserts into the slot, and wherein the second maximum radial displacement is less than the first maximum radial displacement.

Example 22: The medical system of any of examples 17-21, wherein the retrieval structure defines the device recess and the slot, wherein the device recess is configured to receive a first volume of the head section when the protrusion inserts into the device recess and the slot is configured to receive a second volume of the head section when the protrusion inserts into the slot, and wherein the second volume is greater than the first volume.

Example 23: The medical system of any of examples 17-22, wherein the retrieval structure defines the device recess and the slot, wherein the device recess defines a first segment of the structure perimeter and the slot defines a second segment of the structure perimeter, wherein the first segment defines a first segment length and the second segment defines a second segment length, and wherein the second segment length is greater than the first segment length.

Example 24: The medical system of any of examples 17-23, wherein the retrieval structure defines a first slot and a second slot on the structure perimeter, and wherein the retrieval structure defines at least one device recess between the first slot and the second slot on the structure perimeter.

Example 25: The medical system of any of examples 17-24, wherein the retrieval structure defines the slot, and wherein the head section defines a head bearing surface configured to impart the head-transmitted force of example 12 to the slot, and wherein the head bearing surface is configured to be substantially perpendicular to the head-transmitted force of example 12.

Example 26: The medical system of any of examples 17-25, wherein the retrieval structure defines the slot, and wherein the slot defines a slot bearing surface configured to receive the head-transmitted force of example 12, and wherein the slot bearing surface is configured to be substantially perpendicular to the head-transmitted force of example 12.

Example 27: The medical system of any of examples 17-26, wherein a surface defining either the device recess or the device slot is configured to transfer the head-transmitted force of example 12 to the retrieval structure to cause the retrieval structure to rotate about the device axis.

Example 28: The medical system of any of examples 17-27, wherein the implantable medical device includes a fixation element configured to at least one of engage tissue of the patient or disengage from tissue of the patient when the implantable medical device rotates about the device axis.

Example 29: The medical system of any of examples 1-28, further comprising a delivery catheter having a delivery receptacle, wherein the delivery receptacle defines a receptacle volume configured to receive at least a portion of the implantable medical device, wherein the delivery catheter defines a delivery lumen and a delivery lumen opening, wherein the delivery lumen opening opens into the receptacle volume, and wherein the driver body is configured to slidably translate within the delivery lumen and pass through the delivery lumen opening.

Example 30: The medical system of example 29, wherein the receptacle volume is configured to receive the head section and the portion of the implantable medical device when the protrusion inserts into the device recess or the device slot.

Example 31: The medical system of example 30, wherein the driver is configured to rotate relative to the delivery receptacle when the receptacle volume receives the head section and the portion of the implantable medical device, the protrusion inserts into the device recess or the device slot, and the driver imparts the torque on the implantable medical device.

Example 32: The medical system of any of examples 29-31, wherein the delivery catheter is configured transport at least the head section, a portion of the driver body, and a portion of the snare through vasculature of the patient.

Example 33: The medical system of any of examples 1-32, wherein the head section is configured to define the protrusion when the snare causes the head section to exert the contact force on the device recess or the device slot.

Example 34: The medical system of example 33, wherein the head section comprises a material configured to deform to define the protrusion when the snare causes the head section to exert the contact force on the device recess or the device slot.

Example 35: The medical system of example 34, wherein the material is at least one of a thermoplastic elastomer or a liquid silicone rubber).

Example 36: The medical system of example 34 or example 35, wherein the material has a Shore A Hardness greater than or equal to about 10 and less than or equal to about 100.

Example 37: The medical system of any of examples 1-36, wherein the driver includes a driver body supporting the head section.

Example 38: The medical system of example 37, wherein the lumen extends through at least a portion of the driver body.

Example 39: A medical system, comprising: a driver configured to impart a torque on an implantable medical device within an anatomical volume defined by a body of a patient, the driver including a head section, wherein the driver further comprises a lumen extending through at least a portion of the head section, the lumen extending to a lumen opening defined by the head section; and a snare configured to slidably translate within the lumen and through the lumen opening to engage the implantable medical device, wherein the snare is configured to cause the head section to exert a contact force on the implantable medical device when the snare engages the implantable medical device and imparts a force on the implantable medical device, wherein the head section is configured to deform and generate a frictional force with the implantable medical device when the snare causes the contact force and the driver body imparts a torque on the head section, and wherein the head section is configured to transfer the torque to the implantable medical device when the head section generates the frictional force.

Example 40: The medical system of example 39, wherein the material is at least one of a thermoplastic elastomer or a liquid silicone rubber.

Example 41: The medical system of example 39 or example 40, wherein the material has a Shore A Hardness greater than or equal to about 10 and less than or equal to about 100.

Example 42: The medical system of any of examples 39-41, wherein the driver includes a driver body supporting the head section.

Example 43: The medical system of example 42, wherein the lumen extends through at least a portion of the driver body.

Example 44: A medical system, comprising: a snare comprising a snare loop configured to at least partially surround an implantable medical device within an anatomical volume defined by a body of a patient, wherein the snare loop includes a snare surface defining a textured surface; a driver including a driver body having a lumen extending through at least a portion thereof, and a lumen opening that opens to the lumen, wherein the snare extends through the driver lumen and the lumen opening, wherein the snare is configured to constrict around the implantable medical device to exert a snare-imparted force on the implantable medical device when the snare loop at least partially surrounds the implantable medical device and a tension is applied to the snare, wherein the snare surface is configured to frictionally engage the implantable device using the textured surface when the snare loop exerts the snare-imparted force on the implantable medical device, wherein the driver body is configured to exert a force on the snare when the snare surface frictionally engages the implantable medical device, and wherein the snare is configured to impart a torque to the implantable medical device using the frictional engagement when the driver exerts the force on the snare.

Example 45: The medical system of example 44, wherein the lumen defines a longitudinal axis and the tension is substantially parallel to the longitudinal axis.

Example 46: The medical system of example 45, wherein the snare is configured to exert the snare-imparted force in a direction substantially perpendicular to the longitudinal axis.

Example 47: The medical system of example 46, wherein the driver body is configured to cause the snare to exert the snare-imparted force in the direction substantially perpendicular to the longitudinal axis when the driver body is positioned between the snare and the implantable medical device.

Example 48: The medical system of any of examples 44-47, wherein the driver body is configured to exert a driver-imparted contact force to the implantable medical device when the driver is positioned between the snare and the implantable medical device and the snare loop exerts the snare-imparted force on the implantable medical device.

Example 49: The medical system of example 48, wherein the snare is configured to exert the snare-imparted force in a first direction, wherein the driver is configured to exert the driver-imparted contact force in a second direction, and wherein the second direction is opposite the first direction.

Example 50: The medical system of any of examples 44-49, wherein the driver body is configured to exert the force on the snare in a direction substantially perpendicular to the longitudinal axis of example 34.

Example 51: The medical system of any of examples 44-50, wherein the snare is configured to cause the frictional engagement with a retrieval structure of the implantable medical device to impart a torque to the retrieval structure when the driver exerts the force on the snare.

Example 52: The medical system of any of examples 44-51, wherein the snare includes a distal portion including the snare loop and a proximal portion including a snare body, and where the snare loop is configured to exert the snare-imparted force when the the tension is exerted on the snare body.

Example 53: The medical system of any of examples 44-52, wherein the snare surface has a surface roughness at least partially defining the textured surface.

Example 54: The medical system of example 53, wherein the snare surface is configured to cause the surface roughness to produce an engagement force between the snare surface and the implantable medical device when the snare exerts the snare-imparted force.

Example 55: The medical system of any of examples 44-54, wherein the snare surface defines one or more surface undulations at least partially defining the textured surface.

Example 56: The medical system of example 55, wherein at least one surface undulation defines an undulation surface configured to exert a contact force on the implantable medical device when the snare exerts the snare-imparted force and the driver body exerts the force on the snare.

Example 57: The medical system of any of examples 44-56, further comprising the implantable medical device, wherein the implantable medical device defines a retrieval portion, wherein the retrieval portion defines a device axis and a retrieval portion perimeter surrounding the device axis, wherein the snare is configured to at least partially surround the device axis when the snare constricts around the implantable medical device, and wherein the snare is configured to frictionally engage at least a portion of the retrieval portion perimeter using the textured surface.

Example 58: The medical system of example 57, wherein the portion of the retrieval portion perimeter includes one or more line segments, wherein the one or more line segments include at least one of a straight line segment, a curved line segment, or a curvilinear line segment.

Example 59: The medical system of example 57 or example 58, wherein the retrieval portion defines a retrieval portion protrusion defining the portion of the retrieval portion perimeter, where the retrieval portion protrusion is configured to frictionally engage with the snare surface when snare frictionally engages the portion of the retrieval portion perimeter.

Example 60: The medical system of example 59, wherein the portion of the retrieval portion perimeter defines a curve, and wherein the retrieval portion protrusion defines at least a portion of the curve.

Example 61: The medical system of example 60, wherein the curve is a polygonal curve.

Example 62: The medical system of any of examples 57-61, wherein the retrieval portion defines a plurality of retrieval portion protrusions, wherein each individual retrieval portion protrusion defines an individual portion of the retrieval portion perimeter.

Example 63: The medical system of any of examples 57-62, wherein the retrieval portion perimeter defines a polygon.

Example 64: The medical system of any of examples 46-63, wherein the driver include a driver body defining a distal portion and a proximal portion, and wherein the lumen extends through the distal portion and the proximal portion.

Example 65: The medical system of any of examples 46-64, wherein at least a portion of the driver is configured to position between the snare and the implantable medical device when the snare at least partially surrounds the implantable medical device and the driver body translates in a distal direction relative to the snare.

Example 66: The medical system of any of examples 46-65, wherein the snare is configured such that a proximal force exerted on the snare applies the tension to snare when the snare constricts around the implantable medical device, and wherein the snare-imparted force is a portion of a proximal force exerted on the snare.

Example 67: A method, comprising: engaging an implantable medical device, using a snare extending through a lumen and a lumen opening defined by a driver body of a driver, to cause a head section of the driver to exert a contact force on the implantable medical device; imparting a torque, using the driver body, on the head section, wherein the head section defines a protrusion configured to insert within a device recess or a device slot of the implantable medical device; and transferring, using the head section, the torque from the head section to the implantable medical device when the snare causes the contact force and the protrusion inserts within the device recess or the device slot.

Example 68: The method of example 67, further comprising transferring the torque to a retrieval structure defined by the implantable medical device.

Example 69: The method of example 67 or example 68, further comprising increasing the force exerted on the implantable medical device by the snare to increase the contact force.

Example 70: The method of any of examples 67-69, further comprising decreasing the force exerted on the implantable medical device by the snare to decrease the contact force.

Example 71: The method of any of examples 67-70, further comprising, using the torque imparted on the head section, substantially rolling or slipping the head section around a structure perimeter of the implantable medical device to cause the protrusion to insert within the device recess or the device slot.

Example 72: The method system of any of examples 67-71, wherein the protrusion is one of a plurality of protrusions defined by the head section, and wherein inserting the protrusion into the device recess or the device slot comprises inserting any one of the plurality of protrusions into the device recess or the device slot.

Example 73: The method of any of examples 67-72, further comprising rotating the head section relative to the implantable medical device as the head section exerts the contact force.

Example 74: The method of any of examples 67-73, further comprising imparting, using the head section, a head-transmitted force on the device recess when the protrusion inserts into the device recess and the head section transfers the torque to the implantable medical device.

Example 75: The method of any of examples 67-74, further comprising imparting, using the head section, a head-transmitted force on the device slot when the protrusion inserts into the device slot and the head section transfers the torque to the implantable medical device.

Example 76: A method, comprising: exerting, using a driver including a driver body defining a lumen and a lumen opening, a force on a snare when a snare surface of the snare frictionally engages an implantable medical device, wherein the snare extends through the lumen and the lumen opening, and wherein the driver is positioned between the snare and the implantable medical device, and wherein the snare is configured to constrict around the implantable medical device to exert a snare-imparted force on the implantable medical device when a snare loop of the snare at least partially surrounds the implantable medical device and a tension is applied to the snare; and imparting, using the snare, a torque on the implantable medical device when the driver exerts the force on the snare.

Example 77: The method of example 76, wherein the lumen defines a longitudinal axis and the tension is substantially parallel to the longitudinal axis.

Example 78: The method of example 77, wherein the snare exerts the snare-imparted force in a direction substantially perpendicular to the longitudinal axis.

Example 79: The method of example 78, wherein the driver body causes the snare to exert the snare-imparted force in the direction substantially perpendicular to the longitudinal axis when the driver body is positioned between the snare and the implantable medical device.

Example 80: The method of any of examples 76-79, wherein the driver body exerts a driver-imparted contact force to the implantable medical device when the driver is positioned between the snare and the implantable medical device and the snare loop exerts the snare-imparted force on the implantable medical device.

Example 81: The method of example 80, wherein the snare exerts the snare-imparted force in a first direction, wherein the driver is exerts the driver-imparted contact force in a second direction, and wherein the second direction is opposite the first direction.

Example 82: The method of any of examples 76-81, wherein the driver body exerts the force on the snare in a direction substantially perpendicular to the longitudinal axis.

Example 83: The method of any of examples 76-82, wherein the snare causes the frictional engagement with a retrieval structure of the implantable medical device to impart a torque to the retrieval structure when the driver exerts the force on the snare.

Example 84: The method of any of examples 76-83, wherein the snare includes a distal portion including the snare loop and a proximal portion including a snare body, and where the snare loop exerts the snare-imparted force when the tension is exerted on the snare body.

Example 85: The method of any of examples 76-84, wherein the implantable medical device defines a device axis and a structure perimeter surrounding the device axis, and wherein the snare engages a structure protrusion defined by the structure perimeter when snare surface frictionally engages the implantable medical device.

Example 86: The method of any of examples 76-85, further comprising applying a proximal force to the snare to apply the tension to the snare.

Various examples of the disclosure have been described: Any combination of the described systems, operations, or functions is contemplated. These and other examples are within the scope of the following claims.

1: A medical system, comprising:
a driver configured to impart a torque on an implantable medical device within an anatomical volume defined by a body of a patient, the driver including a head section,
wherein the driver comprises a lumen extending through at least a portion of the head section, the lumen extending to a lumen opening defined by the head section, and
wherein the head section or a protrusion thereof is configured to insert within a device recess or a device slot of the implantable medical device; and
a snare configured to slidably translate within the lumen and through the lumen opening to engage the implantable medical device,
wherein the snare is configured to cause the head section to exert a contact force on the implantable medical device when the snare engages the implantable medical device and imparts a force on the implantable medical device, and
wherein the head section is configured to transfer the torque from a driver body of the driver to the implantable medical device when the snare causes the contact force and the protrusion inserts within the device recess or the device slot.

2: The medical system of claim 1, wherein the lumen defines a longitudinal axis, and wherein the head section defines a head perimeter surrounding the longitudinal axis, and wherein the protrusion defines at least a portion of the head perimeter.

3: The medical system of claim 2, wherein the snare is configured to cause the head section to exert the contact force as the head section rotates about the longitudinal axis.

4: The medical system of claim 1, wherein the protrusion is configured to transmit the torque to a retrieval structure defined by the implantable medical device.

5: The medical system of claim 1, wherein the snare is configured to at least one of:
cause the head section to increase the contact force when the snare increases the force exerted on the implantable medical device, or
cause the head section to decrease the contact force when the snare decreases the force exerted on the implantable medical device.

6: The medical system of claim 1, wherein when the driver body imparts a torque on the head section and the head section imparts the contact force on the implantable medical device, the head section is configured to at least one of substantially roll or slip around a structure perimeter of the implantable medical device to cause the protrusion to insert within the device recess or the device slot, wherein the head section exhibits a relative rotation with respect to the implantable medical device when the head section substantially rolls or slips around the structure perimeter.

7: The medical system of claim 1 wherein the protrusion is configured to at least one of:
impart a head-transmitted force on the device recess when the protrusion inserts into the device recess and the head section transfers the torque from the driver body to the implantable medical device, or
impart the head-transmitted force on the device slot when the protrusion inserts into the device slot and the head section transfers the torque from the driver body to the implantable medical device.

8: The medical system of claim 7, wherein the head section is configured to exert the head-transmitted force in a direction substantially perpendicular to a longitudinal axis of the lumen when the driver delivers the torque in a rotational direction around the longitudinal axis of the lumen.

9: The medical system of claim 1, wherein the head section defines a head perimeter, wherein at least a portion of the head perimeter defines a polygonal curve, and wherein the protrusion defines at least a portion of the polygonal curve.

10: The medical system of claim 1, further comprising the implantable medical device,
wherein the implantable medical device includes a retrieval structure defining at least one of the device recess or the device slot,
wherein the retrieval structure defines a device axis and a structure perimeter surrounding the device axis, and
wherein the at least one of device recess or the device slot defines at least a portion of the structure perimeter.

11: The medical system of claim 10,
wherein the retrieval structure defines the device recess and the slot,
wherein the slot defines a first minimum radial displacement from the device axis and the device recess defines a second minimum radial displacement from the device axis, and
wherein the first minimum radial displacement is less than the second minimum radial displacement.

12: The medical system of claim 10,
wherein the retrieval structure defines the device recess and the slot,
wherein the head section defines a first maximum radial displacement from the device axis when the protrusion inserts into the device recess and defines a second maximum radial displacement when the protrusion inserts into the slot, and
wherein the second maximum radial displacement is less than the first maximum radial displacement.

13: The medical system of claim 10, wherein the retrieval structure defines a first slot and a second slot on the structure perimeter, and wherein the retrieval structure defines at least one device recess between the first slot and the second slot on the structure perimeter.

14: The medical system of claim 1, wherein the driver includes a driver body supporting the head section.

15: The medical system of claim 1, wherein the lumen extends through at least a portion of the driver body.

16: A medical system, comprising:
a snare comprising a snare loop configured to at least partially surround an implantable medical device within an anatomical volume defined by a body of a patient, wherein the snare loop includes a snare surface defining a textured surface;
a driver including a driver body having a lumen extending through at least a portion thereof, and a lumen opening that opens to the lumen,
wherein the snare extends through the driver lumen and the lumen opening, wherein the snare is configured to constrict around the implantable medical device to exert a snare-imparted force on the implantable medical device when the snare loop at least partially surrounds the implantable medical device and a tension is applied to the snare,
wherein the snare surface is configured to frictionally engage the implantable device using the textured surface when the snare loop exerts the snare-imparted force on the implantable medical device,
wherein the driver body is configured to exert a force on the snare when the snare surface frictionally engages the implantable medical device, and
wherein the snare is configured to impart a torque to the implantable medical device using the frictional engagement when the driver exerts the force on the snare.

17: The medical system of claim 16,
wherein the driver body is configured to exert a driver-imparted contact force to the implantable medical device when the driver is positioned between the snare and the implantable medical device and the snare loop exerts the snare-imparted force on the implantable medical device, and
wherein the snare is configured to exert the snare-imparted force in a first direction, and
wherein the driver is configured to exert the driver-imparted contact force in a second direction opposite the first direction.

18: The medical system of claim 16, further comprising the implantable medical device,
wherein the implantable medical device defines a retrieval portion,
wherein the retrieval portion defines a device axis and a retrieval portion perimeter surrounding the device axis,
wherein the snare is configured to at least partially surround the device axis when the snare constricts around the implantable medical device, and
wherein the snare is configured to frictionally engage at least a portion of the retrieval portion perimeter using the textured surface.

19: A method, comprising:
engaging an implantable medical device, using a snare extending through a lumen and a lumen opening defined by a driver body of a driver, to cause a head section of the driver to exert a contact force on the implantable medical device;
imparting a torque, using the driver body, on the head section, wherein the head section defines a protrusion configured to insert within a device recess or a device slot of the implantable medical device; and
transferring, using the head section, the torque from the head section to the implantable medical device when the snare causes the contact force and the protrusion inserts within the device recess or the device slot.

20: The method of claim 19, further comprising transferring the torque to a retrieval structure defined by the implantable medical device.
